(19) United States
(12) Patent Application Publication
(10) Pub. No.: US 2022/0170105 A1
(43) Pub. Date: Jun. 2, 2022

(54) METHODS FOR DIAGNOSIS AND PROGNOSIS OF PROSTATE CANCER

(71) Applicant: Phenotype Diagnostics AB, Umeå (SE)

(72) Inventors: Pernilla Wikström, Umeå (SE); Anders Bergh, Umeå (SE); Elin Thysell, Umeå (SE)

(73) Assignee: Phenotype Diagnostics AB, Umeå (SE)

(21) Appl. No.: 17/432,622

(22) PCT Filed: Feb. 21, 2020
(86) PCT No.: PCT/EP2020/054682

§ 371 Date: Aug. 20, 2021

(30) Foreign Application Priority Data
Feb. 22, 2019 (SE) 1950232-7

(51) Int. Cl. C12Q 1/6886

(52) U.S. Cl. CPC C12Q 1/6886; C12Q 2600/112; C12Q 2600/156; C12Q 2600/106; C12Q 2600/158

(57) ABSTRACT

The invention relates to methods for determining tumor aggressiveness and molecular subtype of metastases that are present, or that may eventually develop, in a subject diagnosed with prostate cancer. The methods of the invention comprise determining the molecular subtype of a sample by evaluating levels of subtype-associated gene transcripts. The invention further relates to methods for determining the metastatic potential in a subject diagnosed with prostate cancer and having a primary tumor, as well as to methods for determining the treatment for a subject diagnosed with prostate cancer metastasis.

### TECHNICAL FIELD

The invention relates to methods for determining tumor aggressiveness and molecular subtype of metastases that are present, or that may eventually develop, in a subject diagnosed with prostate cancer. The methods of the invention comprise determining the molecular subtype of a sample by evaluating levels of subtype-associated gene transcripts. The invention further relates to methods for determining the metastatic potential in a subject diagnosed with prostate cancer and having a primary tumor, as well as to methods for determining the treatment for a subject diagnosed with prostate cancer metastases.

### BACKGROUND ART

Bone metastatic disease is the lethal end-stage of aggressive prostate cancer (PC). Patients with metastatic PC are generally treated with androgen deprivation therapy (ADT). This initially reduces metastasis growth, but after some time castration resistant PC (CRPC) develops. Although several new treatments for CRPC have become available they only temporarily retard disease progression (1). Therapy-selection in individual patients as well as future therapeutic developments need to be guided by deeper understanding of bone metastasis biology. This can probably not be obtained by studying primary tumors only or metastases at other locations, since metastases phenotypically diverge due to clonal expansions under the profound influence of different micro-environments, resulting in site-dependent responses to treatment (2, 3).

From studies of the transcriptome and proteome of bone metastases from patients, marked differences between metastases and primary tumors have been identified. Furthermore, metastasis subgroups of apparent biological importance have been identified (4-9). Based on gene expression of canonically AR-regulated genes, 80% of the examined PC bone metastases were defined as AR driven and 20% were defined as non-AR-driven (7). AR-driven bone metastases had high sterol biosynthesis, amino acid and fatty acid degradation, and nucleotide biosynthesis (7), while non-androgen driven metastases showed high immune cell (7) and bone cell activities (8). Proteomic analysis identified two molecular subtypes of bone metastases with different phenotypes and prognosis (9). These observations suggest possibilities for subtype-related treatment of bone metastatic PC.

High proliferation and low tumor cell PSA synthesis in primary PC tumors have been linked to poor prognosis (11, 12, 31-33).

WO 2017/062505 discloses methods for classifying prostate cancer into subtypes. The classification methods may be used to diagnose or prognosticate prostate cancer. In one embodiment, the subtypes are designated PCS1, PCS2, or PCS3. The PCS1 subtype is most likely to progress to metastatic disease or prostate cancer specific mortality when compared to the PCS2 subtype or PCS3 subtype.

However, there is a need for improved methods for determining tumor aggressiveness and molecular subtype of metastases, such as bone metastases, that are present, or that may eventually develop, in a subject diagnosed with prostate cancer. There is also a need for improved methods for patient stratification when selecting the most appropriate therapy for patients with metastatic prostate cancer.

### BRIEF DESCRIPTION OF THE DRAWINGS

FIG. 1. Principal component analysis (PCA) and unsupervised clustering of 72 bone metastasis samples, based on whole genome expression analysis (Illumina bead chip array) identifies three main clusters of samples; MetA, MetB, and MetC. Score plot (a) and loading plot (b) showing MetA-C in black, dark gray and light gray, respectively, based the two first principal components and the clusters in (c). Samples from castration-resistant prostate cancer (CRPC) patients are represented by circles and samples from non-treated and short-term castrated patients are shown as squares. Two neuroendocrine metastases are indicated by stars. Selected sets of gene products enriched in the different metastasis clusters are highlighted. d) Predictions of non-treated, short-term treated, and neuroendocrine samples (gray squares) into clusters defined from PCA analysis of CRPC samples only e) Kaplan-Maier plot showing poor cancer-specific survival for MetB patients after androgen-deprivation therapy (ADT) and f) Top four enriched network categories per metastasis subtype, according to gene set enrichment analysis using the MetaCore software.

FIG. 2. Consistency of metastasis clusters based on the two first principal components for the PCA analysis using five clustering algorithms: i) Hierarchical clustering using the Euclidian distance and Ward linkage, ii) Hierarchical clustering using the Manhattan distance and Ward linkage, iii) k-means clustering, iv) Self Organizing maps and v) Affinity propagation.

FIG. 3. Principal component analysis and orthogonal projections to latent structures discriminant analysis (OPLS-DA) of bone metastasis samples, based on gene expression levels of top 60 differentiating genes for each subtype (see Table 1) showing the score plot, loading plot and hierarchical clusters of a-c) GEO Datasets GSE29650 and GSE101607 and d-f) RNA seq. data (52), g-h) OPLS-DA model for MetA-C based on 72 samples (GSE29650 and GSE101607) and prediction of 43 external samples (yellow) (50), giving frequencies as shown in Table (i).

FIG. 4. Representative tissue sections of MetA, MetB and Met C bone metastases and associated primary tumors stained with HTX-eosin (a-c) and (j-l), PSA (d-f) and (m-o), and Ki67 (g-i) and (p-r). MetA is characterized by moderate cellular atypia, glandular differentiation, relatively low fraction of Ki67 positive cells (proliferating cells) and high PSA immunoreactivity (IR). MetB shows prominent cellular atypia, lack of glandular differentiation, low PSA IR and high tumor cell proliferation. MetC shows prominent cellular atypia with glandular differentiation detectable in some cases, low cell proliferation, relatively low tissue PSA IR, and relatively high stroma/epithelial ratio. MetA associated primary tumors are characterized by high PSA IR and relatively low proliferation. MetB associated primary tumors show low PSA IR, high proliferation, and a reactive stroma response. MetC associated primary tumors show relatively high proliferation with PSA IR and reactive stroma response intermediate between MetA and MetB cases. Bar indicates 100 μm.

FIG. 5. Kaplan-Meier analysis of PSA immunoreactivity (IR) score and proliferation rate (fraction of Ki67-stained tumor cells) in metastasis samples in relation to cancer-specific survival after treatment with androgen-deprivation therapy (ADT). PSA IR was dichotomized as above (high) or below (low) median and Ki67 as quartile 4 (high) or below (low) (a-b). A combinatory PSA and Ki67 score was obtained based on their inverse correlation and the cutoffs used in a-b (c) Patients with high PSA, low Ki67 metastasis IR show the best prognosis with significantly longer cancer-specific survival after first ADT than other patients (d).

FIG. 6. Paired observations of androgen receptor (AR) (a), PSA (b) and Ki67 (c) immunoreactivity (IR) scores in bone metastases of subtypes A-C and in corresponding primary tumor biopsies. The AR and PSA IR were significantly reduced and the proliferation (fraction of Ki67 positive tumor cells) significantly increased in MetA metastases compared to their matched primary tumors.

FIG. 7. Kaplan-Meier analysis of combinatory PSA and Ki67 immunoreactivity (IR) in primary tumor samples in relation to cancer-specific survival after treatment with androgen deprivation therapy (ADT) in metastatic MetA-C patient cohort (a) and in a validation cohort of TUR-P diagnosed patients (b). PSA IR was dichotomized as above (high) or below (low) median and Ki67 as quartile 4 (high) or below (low), using cut-off values for the corresponding cohort. a) Patients with high PSA, low Ki67 primary tumor IR show significantly longer cancer-specific survival after first ADT than other patients. b) Patients with high PSA, low Ki67 show longer and patients with low PSA, high Ki67 show shorter cancer-specific survival after first ADT compared to other patients. c-d) Multivariate Cox analysis shows that the combinatory PSA, Ki67 IR scores evaluated in primary tumors add prognostic value to Gleason scores in metastatic (c) and TUR-P (d) patient cohorts.

FIG. 8. Kaplan-Meier survival analysis of PSA immunoreactivity (IR) (a-b) and a combinatory immunoreactivity (IR) score for PSA and Ki67 (c-f) in relation to cancer-specific survival of patients diagnosed at TUR-P and managed by watchful-waiting. a, c, e) All patients in the cohort and b, d, f) Patients diagnosed with GS≤6 tumors. PSA IR was dichotomized by the median value 9 as high (IR=12) or low (<12). Ki67 was dichotomized by cut-off value for the median (c, d) as Ki67 med-high (Ki67≥2.7%) or Ki67 med-low (<2.7%) or the highest quartile (e, f) as Ki67 Q4-high (Ki67≥5.4%) or Ki67 Q4-low (<5.4%).

FIG. 9. Sensitivity (black) and specificity (grey) for Ki67 (a) and PSA (b) tumor immunoreactivity in identifying death from prostate cancer at different cut-off scores. Patients were diagnosed at TUR-P (1975-1991) and managed by watchful waiting. Median (PSA and Ki67) and Q4 (Ki67) levels for the TUR-P cohort are indicated. The −log (P) values for Cox regression survival analysis using the indicated cut-off values are given in grey.

FIG. 10. A) Predicted frequencies of the MetA-C subtypes in an external cohort of metastases obtained from prostate cancer patients prior to treatment for castration-resistance (52). The OPLS-DA model were based on levels of 157 MetA-C-associated transcripts in the original 72 samples (GSE29650 and GSE101607) and applied on 332 external samples. B) Frequencies of the predicted metastasis subtypes according to metastasis sites.

FIG. 11. A) Serum PSA levels at diagnosis in prostate cancer patients (n=269) in external cohort with metastases of predicted molecular subtypes (52). B) Kaplan-Meier analysis of predicted MetA-C subtypes in relation to patient prognosis after AR-targeting therapy of castration-resistant prostate cancer (n=99).

FIG. 12. Androgen receptor activity (AR, A) and proliferation (B) scores in metastases from castration-resistant prostate cancer patients (52) in relation to predicted molecular subtypes MetA-C (n=332). The scores were calculated from expression levels of predefined AR regulated genes (7) and genes included in the Prolaris gene panel (51).

FIG. 13. Kaplan-Meier analysis of metastasis subtypes in relation to survival after AR targeted therapy for metastatic prostate cancer. Metastasis subtypes in original cohort were defined from unsupervised cluster analysis based on 157 MetA-C associated gene transcripts (A), 428 (B) or 37 (C) PCS1-3-associated gene transcripts (18) or the 157 panel reduced to 113 (D) by removing transcripts redundant with the PCS1-3 panels.

FIG. 14. Kaplan-Meier analysis of predicted metastasis subtypes in the original 72 samples (GSE29650 and GSE101607) in relation to patient survival after androgen deprivation therapy (ADT) for metastatic prostate cancer. The OPLS-DA prediction model was based on levels of (A) 157, (B) 100, (C) 115 and (D) 71 MetA-C-associated transcripts, selected from Table 1 based on different criteria as described in Example 14.

### DESCRIPTION OF THE INVENTION

Three molecular subtypes of PC bone metastases, named MetA, MetB and MetC, have been identified. The said subtypes are related not only to disease outcome, but also to morphology and phenotypic characteristics, and are suggested to be of high clinical significance. Treatment naive and CRPC metastases are found within all subtypes, suggesting that factors other than hormone treatment history are key determinants of subgroup identity. The clinically most contrasting subtypes, MetA and MetB, show characteristics similar to the two subgroups BM1 and BM2, respectively, recently identified by proteome profiling of metastasis samples (9). Furthermore, MetA-C show features resembling subtypes recently described for localized prostate tumors; prostate cancer subtype 1-3 (PCS1-3) (18) and luminal A, luminal B and basal subtypes as determined by the PAM50 breast cancer test (19). Importantly, however, the top 180 differentiating gene products for MetA-C, respectively, and the functionally enriched gene products, show minor overlap with the 428 (18) and 50 (19) biomarkers suggested to differentiate primary tumors into molecular subtypes and with biomarkers on approved tests for predicting risk and selecting therapy in patients with localized disease (Prolaris, OncotypeDx, GenomeDx) (51), with a total overlap of only 46/180 gene products (25%). Based on analysis of MetA-C-associated gene transcripts, the MetA-C subtypes were predicted in external validation cohorts (50, 52) at frequencies comparable to those originally observed, The gene transcripts in Table 1 performed better than biomarkers disclosed in WO 2017/062505 in identifying clinical relevant subgroups of metastases differentiating patients based on response to ADT (see Example 13).

The most common metastasis subtype (MetA) seems to be of luminal cell origin, according to expression of luminal cell differentiation markers and androgen-stimulated genes, including KRT18, FOXA1 and KLK3 (PSA), and signs of glandular differentiation. MetA patients have high serum PSA levels and show good prognosis after ADT. The phenotype of MetA thus resembles that of luminal prostate epithelium.

The MetB subtype shows some features similar to neuroendocrine tumors, such as low AR signaling and high cell cycle and DNA damage response (20), but chromogranin expression is generally low and KRT18 expression retained, suggesting luminal dedifferentiation. The contrasting processes of cell differentiation and proliferation are both driven by androgens in the prostate (21-23), but in a context dependent way that seems reprogrammed during cancer progression by coactivators and corepressors modulating the AR cistrome (24, 25). AR activation in the presence of coactivator FOXA1 results in cell differentiation, PSA secretion and suppressed proliferation (21-23, 26), while in cells with low FOXA1 this instead stimulates cell proliferation (23). In the MetB subtype, androgen-stimulated gene expression is generally low, tumor cells are dedifferentiated, and cell proliferation is high, in parallel with transcript levels of the proliferation-associated transcription factor FOXM1. FOXM1 is known to initiate mitosis (17) and FOXM1 inhibition has been shown to retard tumor growth in a model system for the PCS1 subtype (27).

In the current study, approximately 15% of the samples showed an intermediate subtype with characteristics of both MetA and MetB and in the external cohort (50) this was observed in about 9%. In the LNCaP cell line with a general gene expression pattern similar to PCS2 primary tumors (18), single cell sequencing has demonstrated the existence of multiple sub-clones where some appear similar to MetA whereas others are more MetB-like with high cell proliferation and reduced androgen dependency (28). Collectively, this suggest that the luminal-derived MetA subtype may be able to dedifferentiate in to the more aggressive MetB subtype, possibly driven by altered expression of AR co-regulators such as FOXA1 and/or FOXM1.

The relatively uncommon subgroup MetC is identified based on enrichment of transcripts involved in stroma-epithelial interactions such as cell adhesion, cell and tissue remodeling, immune responses and inflammation. Processes in MetC thus resembles those previously described by us for non-AR-driven bone metastases (7, 8) and for PCS3/basal-like primary tumors of presumed basal cell origin (18, 19). One suggested upstream regulator of MetC is the C/EBP transcription factor, generally associated with inflammation and down-regulated by AR signaling (29). C/EBP is anti-apoptotic and causes chemo-resistance in CRPC, and thus constitutes a potential therapeutic target (29). The stroma fraction in MetC is higher than in MetA and, although this is repeatedly observed in separate metastases of MetC patients, it remains to be shown to what extent the molecular characteristics of MetC is a consequence of lower epithelial content or a key marker of a clearly different tumor phenotype. Furthermore, the cellular origin of MetC and surrogate markers for this apparently multi-faced metastasis phenotype remains to be discovered.

Apparently, the MetA-C subtypes can be determined by other means than by complex molecular profiling. MetB and corresponding primary prostate biopsies are characterized by tumor cell proliferation and dedifferentiation, easily identified by high Ki67 and low PSA immunostaining or by high MCM and low PSA, as recently suggested for BM2 (9). This markedly aggressive phenotype could thus probably be suspected simply by analyzing few surrogate markers, similarly to what is regularly done in breast cancer (30). High proliferation and low tumor cell PSA synthesis in primary PC tumors have been linked to poor prognosis (11, 12, 31-33), but have not previously been combined for prognostication.

When molecular drivers for different metastasis subtypes have been defined, subtype-related treatments could be developed. If androgen signaling promote cell differentiation and inhibit proliferation in subsets of metastases, as could be the case in MetA, ADT may in some cases have adverse effects and additional metabolic targeting could be an option. In other cases, such as MetB patients, ADT should probably be complemented upfront with i.e. chemotherapy, or by direct targeting of tumor promoting factors driving the cell cycle or DNA repair. Patients with MetB bone metastases have reduced AR levels and morphological signs of a reactive stroma response already in their primary tumor stroma, something that has been previously associated with poor response to ADT and poor prognosis (15). For those cases, stroma targeted therapies could be of interest. In breast cancer, responsiveness to hormonal therapy seems to be regulated by signals in the cancer stroma as stroma interfering was able to convert basal, hormone treatment-resistant breast cancer into a luminal, treatment-responsive subtype (34, 35). For MetC patients, potential therapeutic targets in the tumor micro-environment may already be available, such as immune and bone cells.

In conclusion, bone metastases in prostate cancer patients can be separated into at least three molecular subtypes with different morphology, phenotype and outcome. Those subtypes may benefit from different treatments and can be identified by analyzing surrogate markers in metastases, in primary tumors and possibly in liquid biopsies mirroring the whole tumor burden in a patient.

In one aspect, the invention provides a diagnostic method for classifying a prostate cancer subtype in a sample, said sample comprising tumor-derived material from a subject diagnosed with prostate cancer, said method comprising:
(a) obtaining a gene expression profile from the sample;
(b) comparing the obtained gene expression profile with a reference gene expression subtype profile selected from:
   (i) subtype MetA,
   (ii) subtype MetB, and
   (iii) subtype MetC; and
(c) on basis of similarity found in the comparison, classifying the sample as prostate cancer subtype MetA, MetB or MetC.

The term “tumor-derived material” means a material which comprises tumor cells or derivatives thereof. Preferably, the tumor-derived material consists of, or comprises, tumor cells. However, tumor-derived material also includes RNA and protein. The tumor-derived material can preferably be derived from the tumor as such. Alternatively, tumor-derived material can be derived from blood or urine from a subject having a tumor. The said tumor can be a primary tumor or a metastasis, such as a bone metastasis.

The term “sample” means matter that is gathered from the body with the purpose to aid in the process of a medical diagnosis and/or evaluation of an indication for treatment, further medical tests or other procedures. The said sample is preferably obtained by biopsy. A “biopsy” is a medical test involving extraction of sample cells or tissues for examination to determine the presence or extent of a disease. The sample can e.g. be analyzed chemically and/or examined under a microscope. The biopsy can e.g. be an incisional biopsy wherein a portion of abnormal tissue is extracted without removing the entire lesion or tumor. Alternatively, the biopsy can be e.g. a liquid biopsy where tumor-derived material is obtained from a blood or urine sample.

Subtypes MetA, MetB and MetC are defined, for instance, as the prostate cancer subtype which is characterized by expression of a substantial number of the 60 differentiating gene transcripts per subtype shown under “MetA”, “MetB” or “MetC”, respectively, in Table 1. The term “differentiating gene transcripts” means gene transcripts showing significantly higher levels in one subtype compared to the other subtypes, as determined by the prediction model described below in “Experimental Methods”. In the present context, the term “substantial number” can mean a number of at least 10, such as any integer from 10 to 60. The term “substantially all” can mean a number of at least 50, such as any integer from 50 to 60.

A combination of substantially all of the differentiating genes allows for the most accurate classification of the prostate cancer subtype. However, a subtype can be classified on basis of the identification of at least 10 per subtype, such as at least 15, 20, 25, 30, 35, 40, 45, 50 or 55 of the genes shown under “MetA”, “MetB” or “MetC”, respectively, in Table 1.

Consequently, in a first preferred aspect the invention provides a diagnostic method for classifying a prostate cancer subtype in a sample, said sample comprising tumor-derived material from a subject diagnosed with prostate cancer, said method comprising:
(a) obtaining a gene expression profile from the sample;
(b) comparing the obtained gene expression profile with a reference gene expression subtype profile selected from:
   (i) subtype MetA, characterized by increased expression compared to MetB and MetC, of at least 10 of the genes selected from the group consisting of ACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1;
   (ii) subtype MetB, characterized by increased expression compared to MetA and MetC, of at least 10 of the genes selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USP1, and ZNF250; and
   (iii) subtype MetC, characterized by increased expression compared to MetA and MetB, of at least 10 of the genes selected from the group consisting of AEBP1, AP1S2, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5; and
(c) on basis of similarity found in the comparison, classifying the sample as prostate cancer subtype MetA, MetB or MetC.

The reference gene expression profiles can be obtained from e.g. bone metastases tissue or primary tumor tissue from prostate cancer patients. Preferably, the reference gene expression subtype profile is constructed from a plurality of samples comparable to the test sample, said plurality representing samples of each subtype MetA, MetB and MetC.

Preferably, obtaining the gene expression profile from the sample comprises measuring the expression of at least 10 genes from each reference gene expression subtype profile. The obtained gene expression profile is preferably compared to all three of the subtypes MetA, MetB and MetC.

Preferably, the comparing step involves using an algorithm to detect statistically significant similarities in gene expression between the gene expression profile and the reference gene expression profile(s). Accordingly, the classifying step preferably involves using an algorithm to assign the gene expression profile to one of the subtypes MetA, MetB or MetC based on the detected statistically significant similarities in gene expression between the gene expression profile and the reference gene expression profile(s). Alternatively, the comparing step involves using an algorithm to calculate the distance between the gene expression profile and the reference gene expression profile(s). In such case, the classifying step preferably involves using an algorithm to assign the gene expression profile to one of the subtypes MetA, MetB or MetC based on the distance between the gene expression profile and the reference gene expression profile(s).

Subtype MetA

In one aspect, subtype MetA is characterized by increased expression of at least 10 of the genes selected from the group consisting of ACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1.

In a further aspect of the invention, subtype MetA is characterized by increased expression of at least 10 (such as at least 15 or 20; or all 22) of the genes selected from the group consisting ofACAA1, ACP6, ACPP, ACSS1, ALDH1A3, CANT1, CDS1, CRELD1, CTBS, DHRS7, H2AFJ, IVD, KLK3, LOC642299, NAAA, PLA2G4F, SC5DL, SEC22C, SEC23B, SLC25A17, SLC4A4, and VPS54.

In a further aspect of the invention, subtype MetA is characterized by increased expression of at least 10 (such as at least 15 or 20, 25, 30; or all 33) of the genes selected from the group consisting of VPS54, LOC642299, VIPR1, KLK3, SC5DL, SLC4A4, PLA2G4F, ACP6, SEC23B, H2AFJ, DHRS7, HPN, SLC25A17, CDS1, SEC22C, ACSS1, SLC37A1, CRELD1, SELT, ACPP, GTF3C1, SLC35A3, NAAA, SLC9A3R1, IVD, SLC9A2, GABARAPL2, ENTPD6, CANT1, ACAAL SECISBP2L, ALDH1A3, and CTBS.

In yet another aspect of the invention, subtype MetA is characterized by increased expression of at least 20 (such as at least 25, 30, 35; or all 38) of the genes selected from the group consisting of ALDH6A1, C9orf91, CDH1, CPNE4, KL4A0251, KLK2, LOC124220, LOC731999, PPAP2A, REXO2, RNF41, SLC39A6, STEAP2, SUOX, TSPAN1, XBP1, ACAA1, ACP6, ACPP, ACSS1, ALDH1A3, CANT1, CDS1, CRELD1, CTBS, DHRS7, H2AFJ, IVD, KLK3, LOC642299, NAAA, PLA2G4F, SC5DL, SEC22C, SEC23B, SLC25A17, SLC4A4, and VPS54.

In yet another aspect of the invention, subtype MetA is characterized by increased expression of at least 20 (such as at least 25, 30, 35, 40, 45; or all 50) of the genes selected from the group consisting of ACAA1, ACP6, ACPP, ALDH1A3, ALDH6A1, ATP2C1, CANT1, CDH1, CDS1, COG3, CPNE4, CRELD1, DHRS7, ENTPD5, ENTPD6, FAM174B, FICD, GREB1, GTF3C1, H2AFJ, HPN, IVD, KL4A0251, KLK2, KLK3, LOC124220, NAAA, NECAB3, NWD1, PLA2G4F, PPAP2A, REXO2, RNF41, SC5DL, SCCPDH, SEC22C, SEC23B, SECISBP2L, SLC25A17, SLC35A3, SLC37A1, SLC39A6, SLC4A4, SLC9A2, SLC9A3R1, STEAP2, SUOX, TSPAN1, VIPR1, and XBP1.

Subtype MetB

In one aspect, subtype MetB is characterized by increased expression of at least 10 of the genes selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USP1, and ZNF250.

In a further aspect of the invention, subtype MetB is characterized by increased expression of at least 10 (such as at least 15, 20, 25; or all 27) of the genes selected from the group consisting of BUB1, CCNB2, CDC2, CDC20, CDC45L, CDCA4, CKS1B, CKS2, DDX39, DEK, ECT2, FAM83D, GAS2L3, HMGB2, KIF11, KIFC1, LIN9, LOC399942, LOC643287, LSM2, MCM2, NCAPG, NUSAP1, PTMA, RFC5, STMN1, and TUBB.

In a further aspect of the invention, subtype MetB is characterized by increased expression of at least 10 (such as at least 15, 20, 25, 30, 35; or all 36) of the genes selected from the group consisting of RFC5, ECT2, DEK, LSM2, GAS2L3, STMN1, MCM7, MDC1, NCAPG, CKS2, LIN9, NUSAP1, CCNB2, TUBB, CDC45L, LOC643287, CDCA4, CDC2, LOC399942, KIFC1, HMGB2, MCM2, PTMA, FAM83D, KIF11, CDC20, KIF20A, CCNB1, CKS1B, DDX39, C1orf135, BUB1, USP1, CENPL, CCNA2, and PHF16.

In yet another aspect of the invention, subtype MetB is characterized by increased expression of at least 20 (such as at least 25, 30, 35; or all 39) of the genes selected from the group consisting of ASPM C12orf48, C6orf173, KIF15, MCM10, MEST, MSH6, OIP5, STIL, TOP2A, TTK, ZNF250, BUB1, CCNB2, CDC2, CDC20, CDC45L, CDCA4, CKS1B, CKS2, DDX39, DEK, ECT2, FAM83D, GAS2L3, HMGB2, KIF11, KIFC1, LIN9, LOC399942, LOC643287, LSM2, MCM2, NCAPG, NUSAP1, PTMA, RFC5, STMN1, and TUBB.

In yet another aspect of the invention, subtype MetB is characterized by increased expression of at least 20 (such as at least 25, 30, 35, 40, 45, 50 or all 53) of the genes selected from the group consisting of ASPM, BUB1, C12orf48, C16orf75, C17orf53, C1orf135, C6orf173, CCNA2, CCNB1, CCNB2, CDC2, CDC20, CDC45L, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LSM2, MAD2L1, MCM10, MCM2, MCM7, MEST, MSH6, NCAPG, NUSAP1, OIP5, PSRC1, RACGAP1, RFC5, STIL, STMN1, TOP2A, TPX2, TTK, TUBB, UBE2C, UNG, and USPJ.

Subtype MetC

In one aspect, subtype MetC is characterized by increased expression of at least 10 of the genes selected from the group consisting of AEBP1, AP1S2, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5.

In a further aspect of the invention, subtype MetC is characterized by increased expression of at least 10 (such as at least 15 or 20; or all 22) of the genes selected from the group consisting of AEBP1, ARHGAP23, ARHGEF6, C10orf54, CDH5, COL6A3, ENG, FERMT2, FNDC1, FXYD5, GAS6, GIMAP4, KL4A1602, LOC730994, MGC4677, MSN, PDGFRB, PECAM1, RASIP1, STOM, UBTD1, and VAMP5.

In a further aspect of the invention, subtype MetC is characterized by increased expression of at least 10 (such as at least 15 or 20, 25, 30; or all 31) of the genes selected from the group consisting of MSN, STOM, ITGA5, C10orf54, VAMP5, RASIP1, ENG, COL6A2, CYYR1, MGC4677, SRPX2, PARVG, FAM176B, GAS6, ARHGEF6, PLCG2, LOC730994, PECAM1, COL6A3, GIMAP4, CDH5, FNDC1, KIAA1602, ARHGAP23, UBTD1, SH3KBP1, FERMT2, AEBP1, PDGFRB, AP1S2, and FXYD5.

In yet another aspect of the invention, subtype MetC is characterized by increased expression of at least 20 (such as at least 25, 30 or 35; or all 38) of the genes selected from the group consisting of the genes BMP1, C1orf54, CD93, CLDN5, COX7A1, DPYSL2, GYPC, ICAM2, JAM3, LYL1, RAB31, SH2B3, STAB1, TCF4, TPM2, TPST2, AEBP1, ARHGAP23, ARHGEF6, C10orf54, CDH5, COL6A3, ENG, FERMT2, FNDC1, FXYD5, GAS6, GIMAP4, KIAA1602, LOC730994, MGC4677, MSN, PDGFRB, PECAM1, RASIP1, STOM, UBTD1, and VAMP5.

In yet another aspect of the invention, subtype MetC is characterized by increased expression of at least 20 (such as at least 25, 30, 35, 40, 45, 50; or all 54) of the genes selected from the group consisting of the genes AEBP1, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, MSN, NAALADL1, NINJ2, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, and VAMP5.

In a further aspect, the invention provides a method for determining tumor aggressiveness in a subject diagnosed with prostate cancer and having a tumor, said method comprising using the method as disclosed above for classifying a sample, said sample comprising tumor-derived material from the subject diagnosed with prostate cancer, as one of the prostate cancer subtypes MetA, MetB and MetC; wherein
(i) a low or moderate tumor aggressiveness is indicated if the sample is classified as a MetA or MetC subtype; and
(ii) a high tumor aggressiveness is indicated if the sample is classified as a MetB subtype.

The invention further provides a method of screening for the likelihood of effectiveness of prostate cancer treatment comprising androgen deprivation therapy and/or androgen receptor targeting therapy, said method comprising using the method as defined above for classifying a sample, said sample comprising tumor-derived material from a subject diagnosed with prostate cancer, as one of the prostate cancer subtypes MetA, MetB and MetC; wherein

(i) if the sample is classified as a MetA subtype, androgen deprivation therapy and/or androgen receptor targeting therapy alone is likely to be effective in the subject; and

(ii) if the sample is classified as a MetB or MetC subtype, androgen deprivation therapy and/or androgen receptor targeting therapy alone is not likely to be effective in the subject and that additional therapy is warranted. When the subtype is MetB, the additional therapy is preferably chemotherapy and/or therapy using DNA repair inhibitors. When the subtype is MetC, the additional therapy is preferably therapy targeting the micro-environment.

The term “androgen deprivation therapy” (ADT) means antihormone therapy aiming at treating prostate cancer. ADT reduces the levels of androgen hormones, with surgery or drugs (chemical castration), to prevent the prostate cancer cells from growing. Chemical castration includes treatment with GnRH/LHRH analogs or antagonists.

The term “androgen receptor targeting therapy” means therapy that include the use of androgen receptor antagonists, such as bicalutamide, enzalutamide, apalutamide, darolutamide, and others under development for treatment of prostate cancer, or steroidogenesis inhibitors such as abiraterone, ketoconazole, galeterone, and others under development for treatment of prostate cancer.

The term “chemotherapy” (often abbreviated to chemo and sometimes CTX or CTx) means a type of cancer treatment that uses one or more anti-cancer drugs (chemotherapeutic agents). Chemotherapy may be given alone or with other treatments, such as surgery, radiation therapy, or biologic therapy.

Taxane chemotherapy, given with prednisone, is a standard treatment for men with metastatic prostate cancer that has spread and is progressing despite hormone therapy. Taxane chemotherapy agents approved for the treatment of advanced prostate cancer include docetaxel (Taxotere®) and cabazitaxel (Jevtana®).

Platinum-based chemotherapy agents including carboplatin (Paraplatin®), cisplatin (Platinol®), and oxaliplatin (Eloxatin®), are known for the treatment of various cancer types, including prostate cancer.

The term “DNA repair inhibitors” means PARP inhibitors and other DNA repair inhibitors under development for treatment of prostate cancer.

The term “tumor micro-environment” means the environment around a tumor, including the surrounding blood vessels, immune cells, fibroblasts, signaling molecules and the extracellular matrix (ECM). The tumor and the surrounding micro-environment are closely related and interact constantly. It is known to the skilled person that the micro-environment can affect how a tumor grows and spreads. For instance, immune cells in the micro-environment can affect the growth and evolution of cancerous cells. Examples of therapies targeting the tumor micro-environment include the use of immunotherapy; radiopharmaceuticals such as radium-223; as well as bisphosphonates and other osteoclast/osteoblast inhibitors.

A further aspect of the invention is a method of screening for the likelihood of effectiveness of prostate cancer treatment comprising chemotherapy and/or therapy using DNA repair inhibitors, said method comprising using the method as defined above for classifying a sample, said sample comprising tumor-derived material from a subject diagnosed with prostate cancer, as one of the prostate cancer subtypes MetA, MetB and MetC; wherein if the sample is classified as a MetB subtype, chemotherapy and/or therapy using DNA repair inhibitors is likely to be effective in the subject.

Yet another aspect of the invention is a method of screening for the likelihood of effectiveness of prostate cancer treatment comprising targeting the tumor micro-environment, said method comprising using the method as defined above for classifying a sample, said sample comprising tumor-derived material from a subject diagnosed with prostate cancer, as one of the prostate cancer subtypes MetA, MetB and MetC; wherein if the sample is classified as a MetC subtype, targeting the tumor micro-environment is likely to be effective in the subject.

A further aspect of the invention is a method of treating prostate cancer in a subject in need thereof, said method comprising:

(a) using the method as defined above for classifying a sample, said sample comprising tumor-derived material from a subject diagnosed with prostate cancer, as one of the prostate cancer subtypes MetA, MetB and MetC; and

(b) administering a prostate cancer treatment to the subject; wherein

(i) if the sample is classified as a MetA subtype, the subject is administered androgen deprivation therapy and/or androgen receptor targeting therapy, preferably as the sole anti-cancer therapy against the prostate cancer;

(ii) if the sample is classified as a MetB subtype, the subject is administered (I) androgen deprivation therapy and/or androgen receptor targeting therapy, in combination with (II) chemotherapy and/or therapy using DNA repair inhibitors; and

(iii) if the sample is classified as a MetC subtype, the subject is administered (I) androgen deprivation therapy and/or androgen receptor targeting therapy, in combination with (II) therapy targeting the tumor micro-environment.

In still a further aspect, the invention provides a kit for classifying a prostate cancer subtype, said kit comprising (a) reagents for detecting at least 10 (such as at least 20, 25, 30, 35, 40, 45 or 50) biomarkers; and

(b) instructions for using the said reagents in an assay for detecting the presence of the biomarkers; wherein the biomarkers are useful for the detection of prostate cancer subtypes MetA, MetB and/or MetC. Preferably, the said kit comprises biomarkers selected from at least one (1, 2 or 3) of the following groups:
(i) biomarkers for the detection of subtype MetA, selected from the group consisting ofACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1;
(ii) biomarkers for the detection of subtype MetB, selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USPJ, and ZNF250; and
(iii) biomarkers for the detection of subtype MetC, selected from the group consisting of AEBP1, AP1S2, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5.

In additional aspects, the invention comprises the following numbered embodiments as disclosed in Swedish patent application No. 1950232-7, from which priority is claimed:
1. A diagnostic method for classifying a prostate cancer subtype in a sample, said method comprising:
   (a) obtaining a sample comprising tumor-derived material from a subject diagnosed with prostate cancer;
   (b) obtaining a gene expression profile for the said test sample;
   (c) comparing the obtained gene expression profile with the gene expression profile from a reference population;
   (d) assigning the test sample to the prostate cancer subtype designated
      (i) MetA, characterized by increased expression of at least 10 of the genes selected from the group consisting ofACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KL4A0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1;
      (ii) MetB, characterized by increased expression of at least 10 of the genes selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USP1, and ZNF250; or
      (iii) MetC, characterized by increased expression of at least 10 of the genes selected from the group consisting of AEBP1, AP1S2, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5.
2. A method for determining tumor aggressiveness in a subject diagnosed with prostate cancer and having a tumor, said method comprising:
   (a) obtaining a sample comprising tumor-derived material from the said subject diagnosed with prostate cancer; and
   (b) using the method of embodiment 1 for classifying the sample as one of the prostate cancer subtypes MetA, MetB and MetC; wherein
      (i) a low or moderate tumor aggressiveness is indicated if the sample is classified as a MetA or MetC subtype; and
      (ii) a high tumor aggressiveness is indicated if the sample is classified as a MetB subtype.
3. A method of screening for the likelihood of effectiveness of prostate cancer treatment comprising androgen receptor targeting therapy, said method comprising:
   (a) obtaining a sample comprising tumor-derived material from a subject; and
   (b) using the method of embodiment 1 for classifying the sample as one of the prostate cancer subtypes MetA, MetB and MetC; wherein
      (i) if the sample is classified as a MetA subtype, androgen receptor targeting therapy is more likely to be effective in the subject; and
      (ii) if the sample is classified as a MetB or MetC subtype, androgen receptor targeting therapy is less likely to be effective in the subject.
4. A method of screening for the likelihood of effectiveness of prostate cancer treatment comprising chemotherapy, said method comprising:
   (a) obtaining a sample comprising tumor-derived material from a subject; and
   (b) using the method of embodiment 1 for classifying the sample as one of the prostate cancer subtypes MetA, MetB and MetC; wherein
      (i) if the sample is classified as a MetA or MetC subtype, chemotherapy is less likely to be effective in the subject; and
      (ii) if the sample is classified as a MetB subtype, chemotherapy is more likely to be effective in the subject.
5. A method of screening for the likelihood of effectiveness of prostate cancer treatment comprising targeting the tumor micro-environment, said method comprising:
   (a) obtaining a sample comprising tumor-derived material from a subject; and
   (b) using the method of embodiment 1 for classifying the sample as one of the prostate cancer subtypes MetA, MetB and MetC; wherein
      (i) if the sample is classified as a MetA or MetB subtype, targeting the tumor micro-environment is less likely to be effective in the subject; and
      (ii) if the sample is classified as a MetC subtype, targeting the tumor micro-environment is more likely to be effective in the subject.
6. A method of treating prostate cancer in a subject in need thereof, said method comprising:
   (a) obtaining a sample comprising tumor-derived material from the said subject;
   (b) using the method of embodiment 1 for classifying the sample as one of the prostate cancer subtypes MetA, MetB and MetC; and
   (c) administering a prostate cancer treatment to the subject; wherein
      (i) if the sample is classified as a MetA subtype, the subject is administered androgen-deprivation therapy in combination with additional therapies targeting the androgen receptor;
      (ii) if the sample is classified as a MetB subtype, the subject is administered androgen-deprivation therapy in combination with chemotherapy; and
      (iii) if the sample is classified as a MetC subtype, the subject is administered androgen-deprivation therapy in combination with therapies targeting the tumor micro-environment.
7. The method according to any one of embodiments 1 to 6 wherein the said tumor-derived material comprises tumor cells.
8. A kit for classifying a prostate cancer subtype, said kit comprising
   (a) reagents for detecting at least 10 biomarkers; and
   (b) instructions for using the said reagents in an assay for detecting the presence of the at least 10 biomarkers;
   wherein the biomarkers are selected from one of the following groups:
      (i) biomarkers for the detection of subtype MetA, selected from the group consisting ofACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1;
      (ii) biomarkers for the detection of subtype MetB, selected from the group consisting of ASPM, BUB1, C12orf48, C16orf75, C17orf53, C1orf135, C6orf173, CCNA2, CCNB1, CCNB2, CDC2, CDC20, CDC45l, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USP1, and ZNF250; or
      (iii) biomarkers for the detection of subtype MetC, selected from the group consisting of AEBP1, AP1S2, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5.

### EXPERIMENTAL METHODS

Patient samples:

Samples of bone metastases were obtained from a series of fresh-frozen and formalin-fixed paraffin embedded (FFPE) biopsies collected from patients (n=72) with PC operated for metastatic spinal cord compression at Umeå University Hospital (2003-2013). Primary tumor biopsies (FFPE) were available in in 52 cases. The patient series and the tissue handling have been previously described (4, 7, 10).

Patients gave their informed consent and the study was conducted in accordance with the Declaration of Helsinki.

Primary tumor samples were also obtained from a historical cohort of 419 men with PCa, detected after transurethral resection of the prostate (TURP) due to voiding symptoms, 1975-1991, in Vasteras, Sweden, for details see (11, 12). Patients with symptomatic metastases were treated with ADT, a few patients were treated with radiation or radical prostatectomy, while a majority of men were followed with expectancy (“watchful waiting”) according to clinical practice at that. All cases were Gleason regraded by a single pathologist.

RNA Extraction and Gene Expression Analysis:

RNA was extracted from representative areas of fresh frozen bone metastases sections using the Trizol (Invitrogen, Carlsbad, CA) or the AllPrep DNA/RNA/Protein Mini Kit (QIAGEN, Hilden, Germany) protocols. Nucleic acids were quantified by absorbance measurements using a spectrophotometer (ND-1000 spectrophotometer; NanoDrop Technologies Inc, Wilmington, Del.). The RNA quality was analyzed with the 2100 Bioanalyzer (Agilent Technologies, Santa Clara, Calif.) and verified to have an RNA integrity number ≥6. Whole genome expression array analysis was performed using the human HT12 Illumina Beadchip technique (Illumina, San Diego, Calif.) with version 3 in (4) and version 4 in (7).

Bead chip data from two separate gene expression studies (GEO Datasets GSE29650 and GSE101607) were combined for all probes with average signals above twice the mean background level in at least one sample per study array. Arrays were individually normalized to remove batch effects, using the quantile method followed by centering of the data by subtracting the mean signal for each probe. Normalized datasets were merged by mapping Illumina ID and Hugo gene symbol. Redundant transcript probes were removed by selecting the probe with the highest median expression, leaving 10784 gene transcripts for subsequent analysis. When merging bead chip data with external RNA sequencing data (50, 52) in class discriminant analysis (below), data was centered by dividing intensities for each gene product by the median in each cohort.

Multivariate Data Analysis:

Principal component analysis (PCA) was used to get an overview of the variability in data and to detect potential subgroups by unsupervised pattern recognition. Sevenfold cross-validation testing was used to assess the reliability of the model. Cluster analysis was performed based on the first m (m=2, 5) principal components, using five clustering algorithms: i) Hierarchical clustering using the Euclidian distance and Ward linkage, ii) Hierarchical clustering using the Manhattan distance and Ward linkage, iii) k-means clustering, iv) Self Organizing maps and v) Affinity propagation (13).

A prediction model for subtype was built using orthogonal projections to latent structures discriminant analysis, OPLS-DA (51), based on levels for the top 60 gene products differentiating one sample cluster from the others (defined by the lowest P values in Mann-Whitney U test and a median fold change ≥1.5), and applied to an external cohort of 43 bone metastases (50). OPLS-DA maximizes the explained variation in data (X) and its covariation with class membership, Y, defined by a dummy matrix of zeros and ones. Class membership was defined as software default, by predicted value i) <0.35 do not belong to the class, ii) between 0.35 and 0.65 intermediate and iii) above 0.65 belong to the class. Multivariate data modelling was performed with SIMCA software version 15.0 (Umetrics AB, Umeå, Sweden). Similarly, a prediction model for subtype classification was built based on the gene products in Table 1 and applied to an external cohort of PC bone metastases with RNA sequencing data available from 332 cases and clinical data available for a fraction of those (52). The predictability of models based on selected gene products in Table 1 were evaluated in the GEO Datasets GSE29650 and GSE101607.

Functional Enrichment Analysis:

Gene set enrichment analysis (GSEA) was performed by the MetaCore software (GeneGo, Thomson Reuters, New York, N.Y.). Analysis was based on gene transcripts significantly increased in one cluster compared to the others, as defined by Kruskal Wallis followed by Mann-Whitney U test and adjusted P values (False Discovery Rate, FDR, <0.01). Sets of genes associated with a functional process (pathway map or network) were determined as significantly enriched per subtype based on P values representing the probability for a process to arise by chance, considering the numbers of enriched gene products in the data vs. number of genes in the process. P values were adjusted by considering the rank of the process, given the total number of processes in the MetaCore ontology. Possible drivers of each subtype were identified by exploring the relations between subtype-enriched transcripts and upstream regulators defined from the literature. P-values were calculated for connectivity ratios between actual and expected interactions with objects in the data.

Metastases and Primary Tumor Morphology:

The fraction of tumor epithelial cells in metastasis tissue was determined using stereological techniques, as earlier described (14). Metastasis cell atypia was graded either as moderate or pronounced and glandular differentiation was scored as observed or not. Cancer cells in metastases and primary tumor biopsies were stained and scored for AR, PSA, Ki67, and chromogranin-A as earlier described (10).

The PSA staining, using the A0562 PSA antibody (Dako) were quantified using a scoring system based on the percentage (0=no staining, 1=1-25%, 2=26-50%, 3=51-75% and 4=76-100% of tumor epithelial cells stained) and intensity (0=no staining 1=week, 2=moderate and 3=intense) of immunostained tumor epithelial cells. An immunoreactivity (IR) score was obtained by multiplying the scores for distribution and intensity, as earlier described (10), giving IR scores in the range of 0-12. Ki67 staining, using the anti-Ki-67 (30-9) Rabbit Monoclonal Primary Antibody (Roche Diagnostics), was quantified as the percentage of stained tumor epithelial cells (10). Combinatory PSA and Ki67 immunoreactivity scores were obtained using cut-offs at median or the upper quartile (per sample cohort), and by this patients were categorized into 4 different groups 1) PSA high/Ki67 low, 2) PSA high/Ki67 high, 3) PSA low/Ki67 low, and 4) PSA low/Ki67 high.

The stroma in primary tumor biopsies was scored for the percentage of AR positive cells as earlier described (15) and for a reactive desmoplastic response, characterized by loss of stroma smooth muscle and increase in fibroblasts and collagen, using a 3-tier scoring system (16).

Univariate Statistics and Survival Analysis:

Continuous variables were given as median (25th; 75th percentiles) and non-parametric statistics was used (Mann-Whitney U test, Wilcoxon test, Spearman rank correlation). The Chi-square test was used for categorical values. Survival analysis was performed by Kaplan-Meier analysis with death of PC as event and death by other causes as censored events and with follow-up time considering time from diagnosis or time from first ADT until the latest follow-up examination. The log-rank test was used to test for statistical significance in differences in survival. Cox proportional hazard models were used and results presented as hazard ratio (HR) with 95% confidence intervals. All tests were two sided and P-value less than 0.05 were considered statistically significant. Statistical analyses were performed using the Statistical Package for the Social Sciences, SPSS 24.0 software (SPSS, Inc, Chicago, USA).

### EXAMPLES OF THE INVENTION

### Example 1: Global Gene-Expression in None Metastases and Identification of Robust Molecular Subtypes

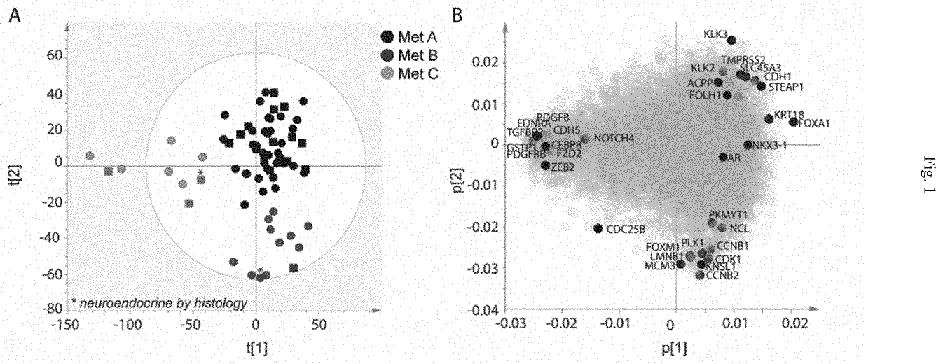

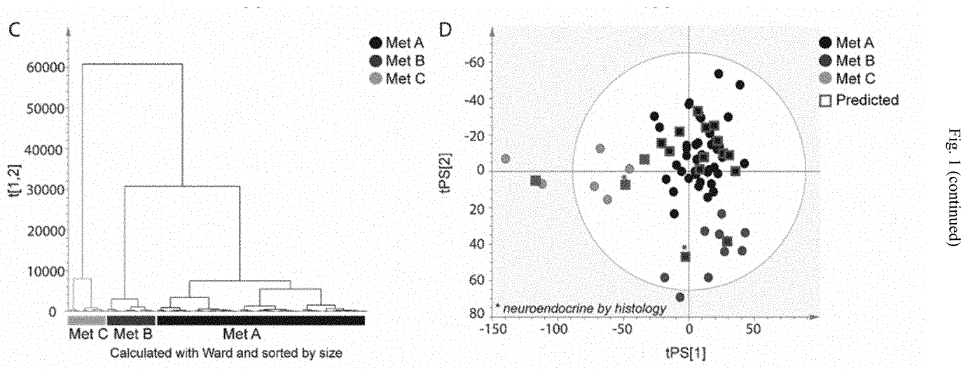

The global gene-expression pattern in 12 treatment-naive, 4 short-term castrated, and 56 CRPC bone metastases was explored. Based on transcript levels of 10784 non-redundant genes, a principal component analysis (PCA) model was built that included 9 significant principal components explaining 40% of the variation in the data. Hierarchical cluster analysis using the Euclidian distance and the first two principal components revealed three molecular subtypes of bone metastasis, referred to as metastasis subtype A, B, and C (MetA-C) (FIG. 1). The majority of samples clustered as MetA (71%), while 17% and 12% clustered as MetB and MetC, respectively (FIG. 1a-c), based on the loadings (gene expression levels) in FIG. 1c.

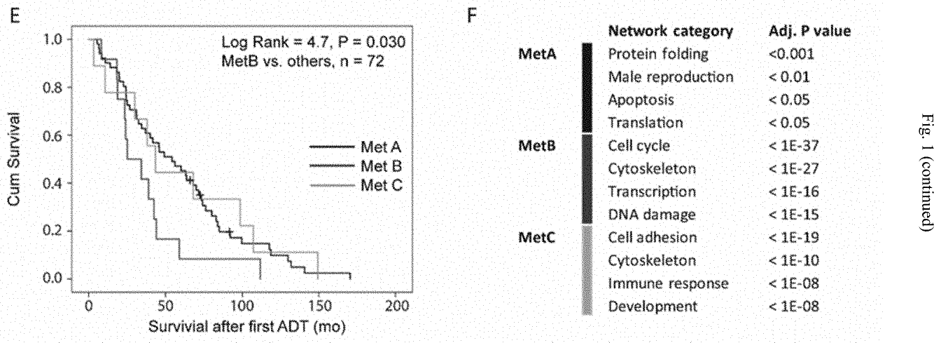

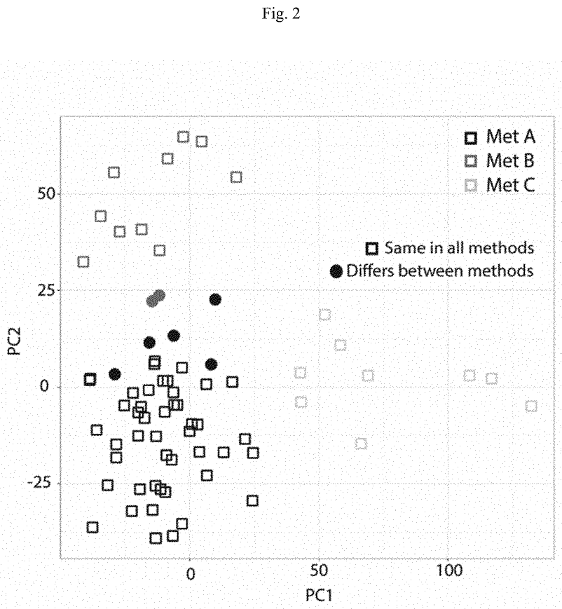

The inclusion of 5 principal components and the use of alternative clustering methods verified robust clustering with preserved grouping of 90% of the samples, and 90%, 83% and 100% consistency for the MetA, MetB and MetC samples, respectively (FIG. 2). Importantly, the MetA-C clusters were identified also when data analysis was based on CRPC samples only (FIG. 1d), leaving samples from treatment-naive and short-term castrated patients outside the PCA modelling together with two CRPC samples defined as neuroendocrine (NE, based on high chromogranin A and low PSA, AR expression). Those samples were predicted with 100% consistency and previously untreated metastases were identified within all clusters (FIG. 1a,d), indicating that the MetA-C subtypes are intrinsic and not developed by the introduction of castration therapy.

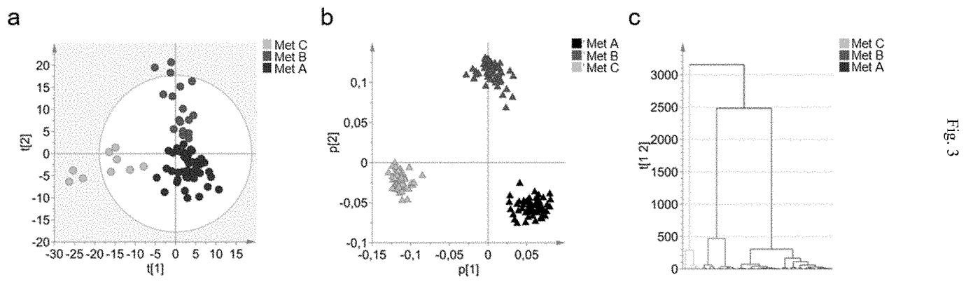

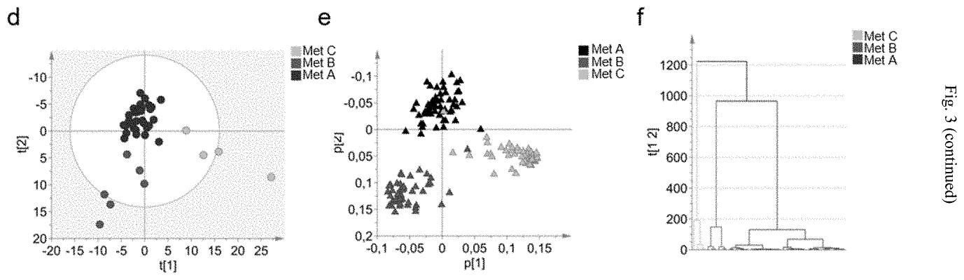

To enable validation of the MetA-C subtypes in an external data set of PC bone metastases (50), the top 60 gene products differentiating each sample cluster from the others (Table 1) were identified and used for PCA and OPLS-DA modelling (FIG. 3). Expression levels for the MetA-, MetB-, and MetC-associated genes, respectively, were highly correlated also within the external cohort and responsible for differentiating samples into three clusters (FIG. 3a-f). Accordingly, the MetA-C subtypes in the validation cohort were predicted at frequencies comparable to those originally observed (FIG. 3g-i).

### Example 2: Metastasis Subtypes Relate to Patient Characteristics and Prognosis

As can be seen in Table 2, most patients were diagnosed with locally advanced or metastatic disease; high serum PSA levels, and poor tumor differentiation (high Gleason score, GS). In patients where PC was not diagnosed until it caused neurological symptoms (patients without ADT at metastasis surgery), the primary tumor was not biopsied. Most patients were directly treated with ADT, while 10 patients had been previously treated with curative intent (Table 2). In 52 cases (72%) there were available primary tumor biopsies for morphological analysis. At relapse to castration resistance, patients had been given second line treatments as indicated (Table 2).

To assess the clinical relevance of the molecular subtypes, MetA-C were analyzed in relation to the patient characteristics summarized in Table 2. Patients with the MetB subtype had shorter cancer-specific survival after ADT than MetA and MetC patients (median survival 25 months vs. 49 months, respectively, P=0.030, FIG. 1e), and lower serum PSA levels compared to MetA patients at diagnosis (0.28-fold, P=0.011) and borderline at metastasis surgery (Table 2). A tendency of low PSA levels was seen also in MetC patients (Table 2). As described above, the subtypes were not related to previous ADT (FIG. 1), while a relatively high proportion of MetB patients had undergone radiation therapy to primary tumor (P=0.006) and received bicalutamide and/or chemotherapy subsequent to ADT (P=0.038 and 0.017, respectively, Table 2). This discrepancy in treatment history may be related to the particularly aggressive clinical course and poor response to ADT in MetB patients (FIG. 1e). Neither primary tumor Gleason score (GS) nor patient age or soft tissue metastasis were significantly associated with any specific subtype.

### Example 3: Metastasis Subtypes have Different Morphology

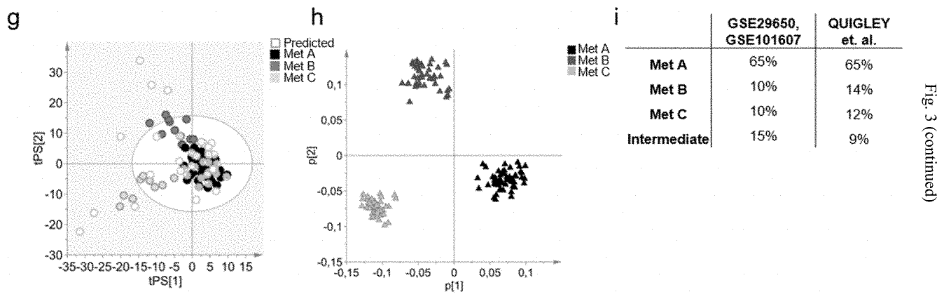

Most metastases were poorly differentiated with sheets of tumor epithelial cells resembling Gleason grade 5, while some showed patterns similar to Gleason grade 4 (FIG. 4a-c). Some metastases showed a prominent connective tissue stroma (FIG. 4a-c). The fraction of cancer cells was significantly lower in MetC compared to MetA tumor sections (Table 2). Importantly, this was seen both in the frozen sections (used for gene-expression analysis) and in the paraffin-embedded tissue (used for morphology analysis) representing distinct metastasis areas from the same patient, suggesting intrinsic differences in epithelium/stroma ratio between subtypes. Additional subtype-related differences were identified based on histological and immunohistochemical analysis of markers previously associated with aggressive PC (summarized in Table 3), with the most pronounced being reduced tissue PSA, increased proliferation (fraction of Ki67-stained tumor cells), cellular atypia and lack of glandular structures in MetB. Marked intra-tumor heterogeneity in immune-staining pattern was observed, as previously reported (10).

### Example 4: Enrichment of Divergent Functional Processes Per Metastasis Subtype

To identify subtype-enriched functional processes, gene transcripts with significantly increased levels per subtype were subjected to GSEA in the MetaCore software. Network analysis showed enrichment of protein translation and folding, male reproduction and regulation of apoptosis in MetA; cell cycle and DNA damage response, cytoskeleton reorganization and transcription in MetB; and cell adhesion, cytoskeleton, immune response, and development in MetC (FIG. 1f).

Pathway analysis demonstrated enrichment of “AR activation and downstream signaling in prostate cancer” in MetA compared to other subtypes, based on high transcript levels of KLK3 and other canonically AR-regulated genes such as KLK2, FOLH1, STEAP1, TMPRSS2, SLC45A3, ACPP (PPRP), and CDH1 (FIG. 1b). MetA also showed high expression of the luminal cell marker KRT18 (FIG. 1b) and enrichment of metabolic pathways involving amino acid and fatty acid degradation.

The MetB subtype showed pathway enrichment representing all phases of the cell cycle, including “Initiation of mitosis”, based on high FOXM1, CCNB1, CCNB2, CDC25B, CDK1, PLK1, PKMYT1, LMNB1, KNSL1, and NCL expression (FIG. 1b) Other markedly enriched pathways in MetB included response to DNA damage and transcription. MetB expression levels of KRT18 were similar to MetA, while most luminal cell markers like as KLK3 and CDH1 were reduced, indicating luminal cell dedifferentiation coupled to increased cell division.

Among many enriched pathways in MetC, “ECM remodeling”, “regulation of EMT”, and “immunological synapse formation” were among the most prominent. Enrichment of “the EMT pathway” in MetC was based on high levels of transcripts involved in Wnt, Notch, TGF-beta, and PDGF signaling (FIG. 1b). MetC showed low expression of luminal cell markers, but was enriched for some transcripts indicating a basal cell phenotype; i.e. CEBPB and GSTP1. Other basal cell markers like p63 and CK5 were low in all cases. Expression levels of luminal cell markers AR and NKX3.1 did not significantly differ between subtypes.

### Example 5: Possible Drivers of Metastasis Subtypes

As the MetB subtype was associated with the worst clinical outcome, putative drivers of its key characteristics, i.e. luminal cell dedifferentiation and proliferation, were identified. Based on connectivity analysis of gene networks and upstream regulators, a set of interesting candidate drivers were identified, such as the FOXA1 transcription factor (HNF3alpha) in MetA and the FOXM1 transcription factor in MetB. While FOXA1 may interact with the AR in MetA to drive canonical AR signaling and luminal differentiation (FIG. 1b), FOXM1 may drive proliferation in MetB (FIG. 1b) (17).

Several kinases with inhibiting drugs available in the clinic for treatment of other cancer types or in clinical trials were suggested as upstream regulators for specific subtypes, e.g. ErbB2 (MetA), AURORA A/B (MetB), and PDGF-R-beta (MetC), hypothetically indicating possibilities for subtype-related therapeutic options.

### Example 6: Immunohistochemistry to Determine Metastasis Subtype

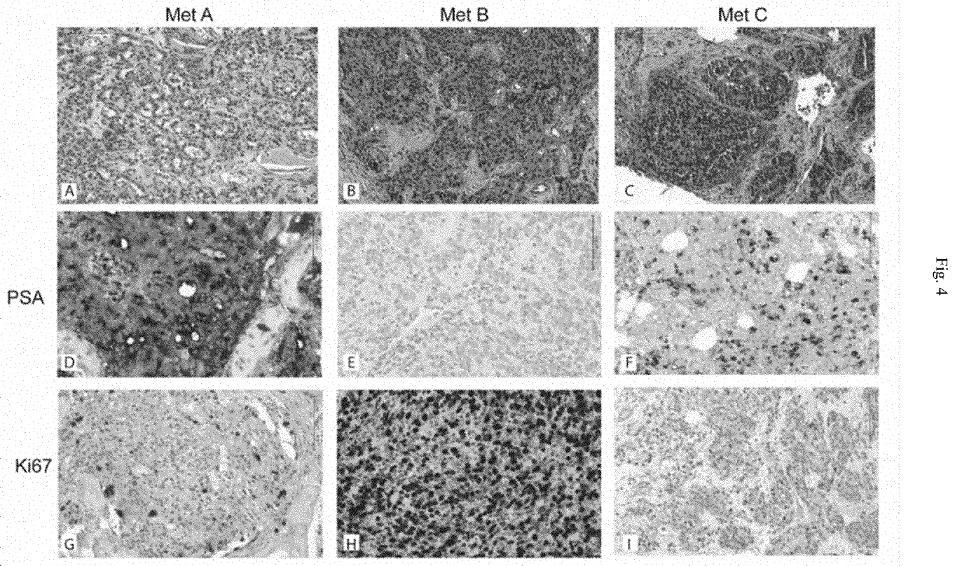

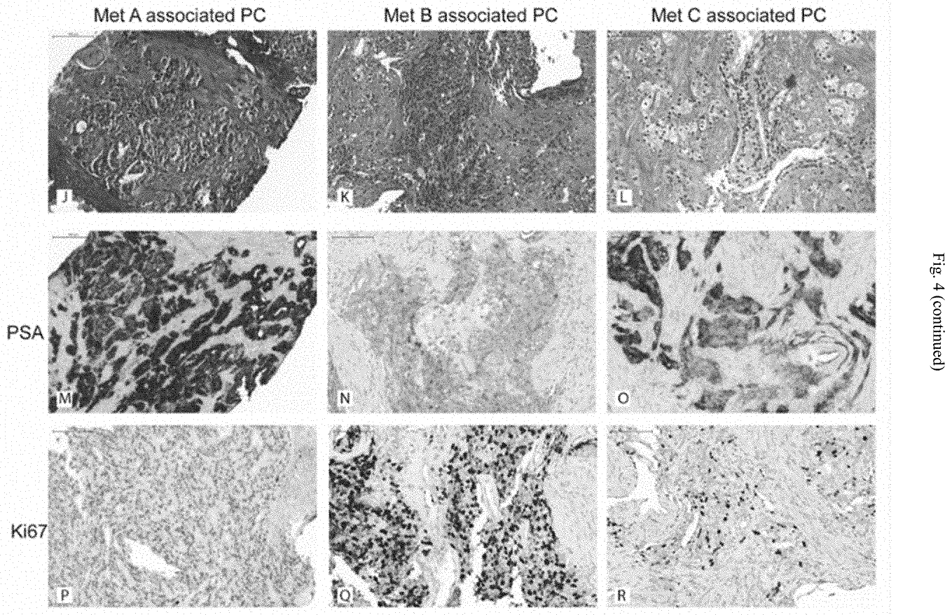

Based on gene expression data and morphological observations, PSA and Ki67 were selected as potential subtype-related surrogate markers (FIG. 4d-i, Table 3). Notably, the PSA staining score was higher in metastases with than without glandular differentiation (P=0.016, n=72) and in cases without pronounced atypia (P=0.012, n=72), suggesting that high cellular PSA is a marker for preserved epithelial and glandular differentiation in tumor cells. Accordingly, patients with low PSA staining scores (below median, scores 0-6) and high proliferation (fraction of Ki67 stained cells in the upper quartile, >25%), respectively, had short cancer-specific survival after first ADT in comparison to other patients (FIG. 5a-b). The PSA staining score inversely correlated to tumor cell proliferation in bone metastases (Rs=−0.32, P=0.007, n=71) (FIG. 5c), and a combinatory score identified 4 groups of metastases with the following frequencies; high PSA, low Ki67 (41%); low PSA, low Ki67 (32%); low PSA, high Ki67 (18%); high PSA, high Ki67 (8.5%) (FIG. 5d). MetB samples were enriched among the low PSA, high Ki67 samples (9/13, 69%) whereas MetC was not specifically enriched by these markers. Patients with high PSA, low Ki67 were enriched for MetA (86%) and showed the best prognosis (FIG. 5d).

### Example 7: Comparisons Between Bone Metastases and Paired Primary Tumors

It was investigated whether subtype-related difference in metastases could be traced back to the corresponding primary tumors, by exploring morphologic factors in diagnostic needle biopsies, as summarized in Table 3 and demonstrated in FIG. 4j-r. Collectively, these observations indicated that characteristics of MetB, such as high proliferation and low tissue PSA, may be detectable already in the primary tumor (Table 3). Primary tumors of MetB patients also showed low AR staining in the tumor stroma coupled to a reactive stroma response (Table 3).

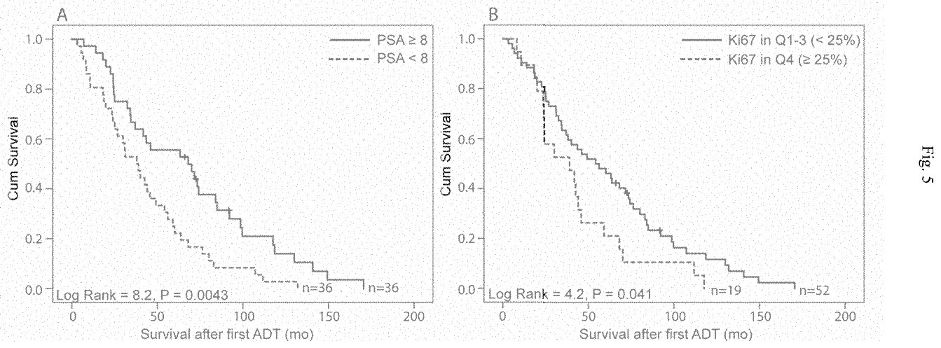

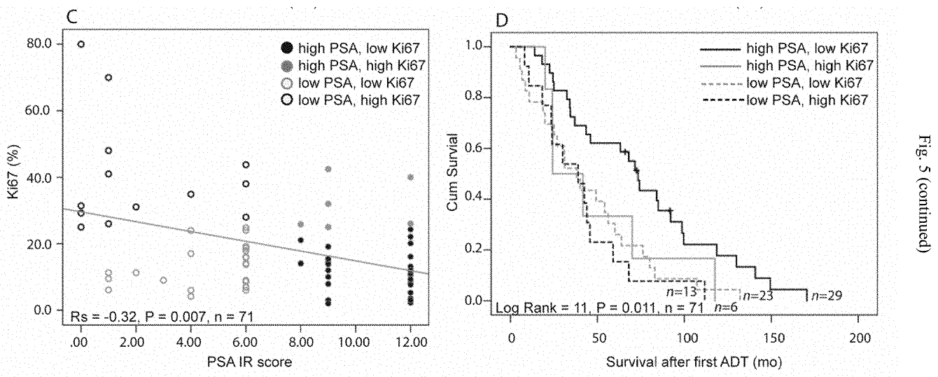

Paired-wise analysis showed significantly reduced AR (P=2.3E-5, n=34) and PSA (P=0.017, n=32) staining in MetA metastases compared to their corresponding primary tumors, while the fraction of Ki67 positive cells was significantly increased (P=0.013, n=35) (FIG. 6). Those markers did not significantly change from primary tumor to metastasis in MetB or MetC patients (FIG. 6).

### Example 8: Determining Prognosis by Analysis of Subtype-Related Markers in Primary Tumors

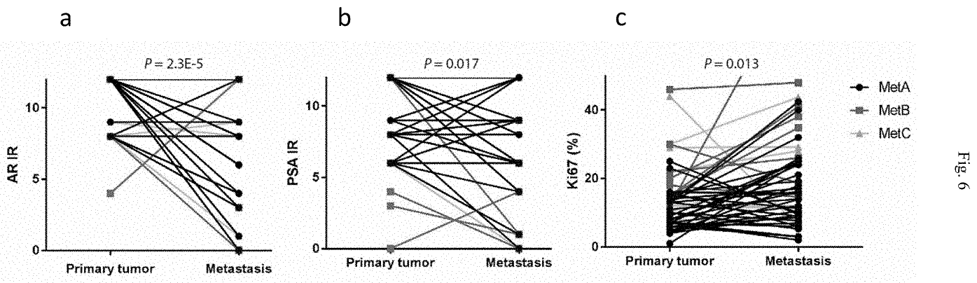

It was investigated whether surrogate immunohistochemical markers for the MetA and MetB phenotypes could differentiate patient outcome also if analyzed in primary tumor tissue. High Ki67 and low PSA immunoreactivity (MetB enriched) was associated with short survival after first ADT in two different cohorts; i) primary tumor biopsies of the MetA-C patients in the current study (FIG. 7a) and ii) TURP diagnosed cases (FIG. 7b), by using the PSA median and Ki67 upper quartile as cut-off values for the corresponding cohort. Patients with the combination of high PSA and low Ki67 (MetA-enriched) had a more favorable outcome than other patients when treated by ADT (FIG. 7a-b). The combinatory PSA and Ki67 IR score provided independent prognostic information to GS in multivariate survival analysis (FIG. 7c-d).

### Example 9: Reduced Tissue PSA Level and Increased Ki67 Labelling are Related to Poor Outcome in Patients Treated with Watchful Waiting

Data were obtained from a historical cohort of men with PCa detected after transurethral resection of the prostate (TURP) due to voiding symptoms. Immunohistochemical data for tumor cell proliferation (fraction of Ki67 positive cells) was available for 389 of the cases (11, 12). The available original tissue blocks were now sectioned and stained for PSA (n=347), as earlier described (10), resulting in combined Ki67 and PSA data in 332 cases. In non-malignant prostate tissue the glandular luminal cells showed intense PSA staining (score 3) in at least 75% of the glandular tissue (score 4), resulting in a PSA IR score of 12. This staining pattern was the most common also in prostate cancers, seen in 48% of the cases. However, in many men reduced PSA staining was seen in parts of or in the entire tumor, giving PSA IR score below 12.

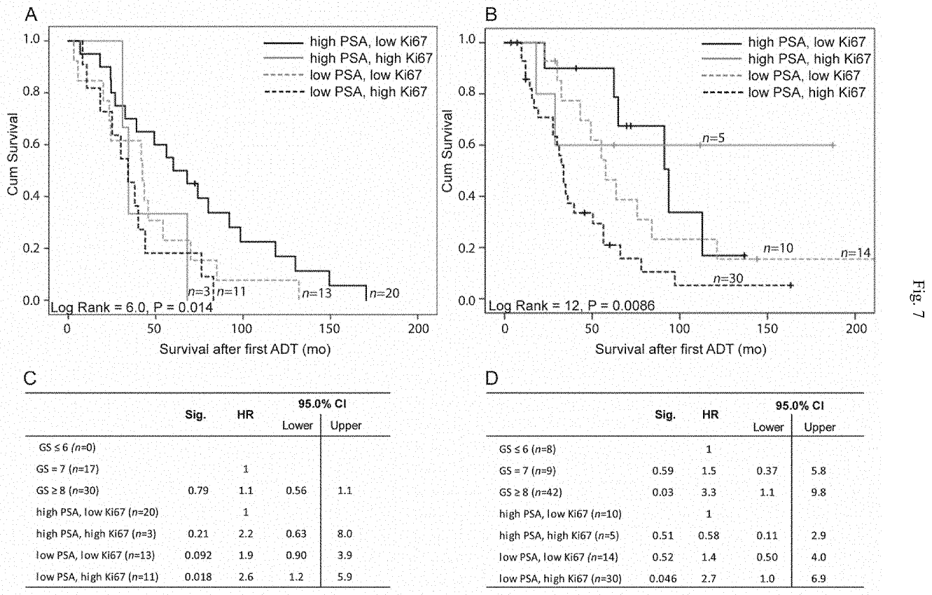

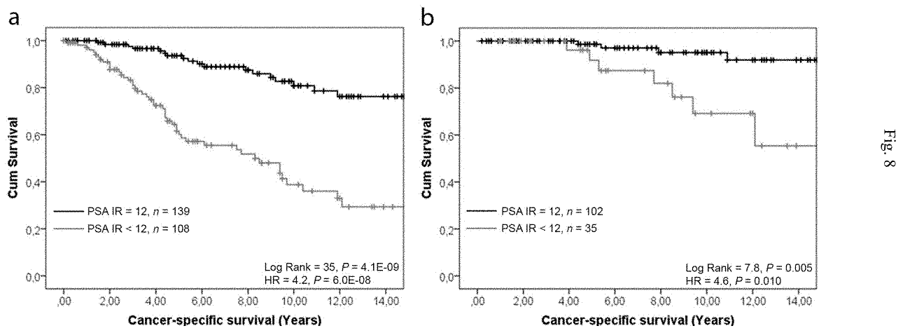

Men managed with watchful waiting and available PSA scores (n=247) were analyzed for cancer specific survival. Patients with a low PSA IR score (below 12) had short cancer specific survival compared to those with a PSA IR score of 12 (FIG. 8a). Specifically, low level of PSA staining was associated with poor prognosis also in men with GS≤6 (FIG. 8b).

In men managed with watchful waiting, increased Ki67 labeling above median and particularly in the highest quartile (Q4) were associated with a poor outcome as earlier described in more detail (11, 12).

### Example 10: Combined Analysis of PSA and Ki67 Immunoreactivity Identifies Patients with Different Prognosis when Treated with Watchful Waiting

The Ki67 and PSA immunostaining scores were moderately and inversely correlated (Spearman rank correlation=−0.46, p<0.001), but both variables provided independent prognostic information from GS in multivariate Cox survival analysis (Table 3). The PSA and Ki67 values were therefore used in combination. First, the median (med) IR scores; PSA (>9) and Ki67 (≥2.7%), were used as cut-off values for “high” levels and to separate tumors into 4 different groups:
(1) PSA high/Ki67 low;
(2) PSA high/Ki67 high;
(3) PSA low/Ki67 low; and
(4) PSA low/Ki67 high.

Kaplan-Meier survival analysis showed that these groups had different outcomes when managed by watchful waiting, with PSA high/Ki67 med-low being the most favorable and PSA low/Ki67 med-high the worst combination (FIG. 8c). This was true also for patients with GS≤6 (FIG. 8d). Patients with PSA high/Ki67 med-high and PSA low/Ki67 med-low cases showed intermediate prognosis.

In order to identify a subgroup of patients with a particularly poor prognosis, patients were divided into PSA/Ki67 groups using Q4 (≥5.4%) as the cut-off value for “Ki67 high”:
(1) PSA high/Ki67 Q4-low (121/237, 51%, of men managed by watchful waiting);
(2) PSA high/Ki67 Q4-high (11/237, 4.6%);
(3) PSA low/Ki67 Q4-low (78/237, 33%); and
(4) PSA low/Ki67 Q4-high (27/237, 11%).

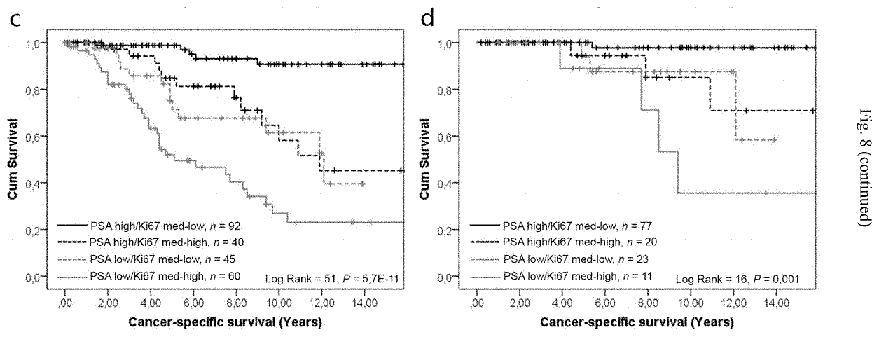

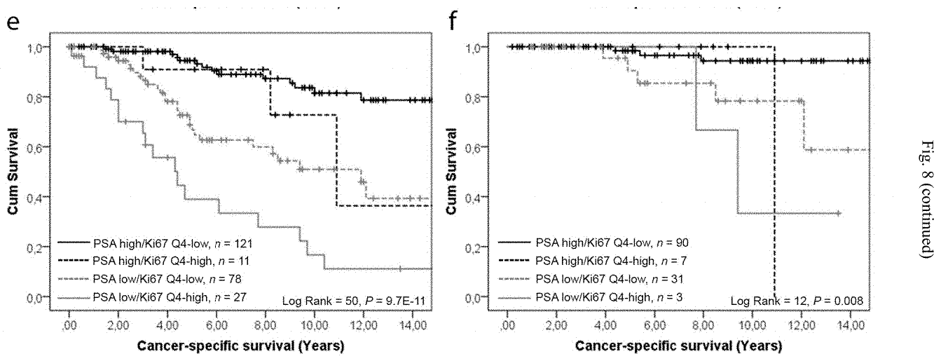

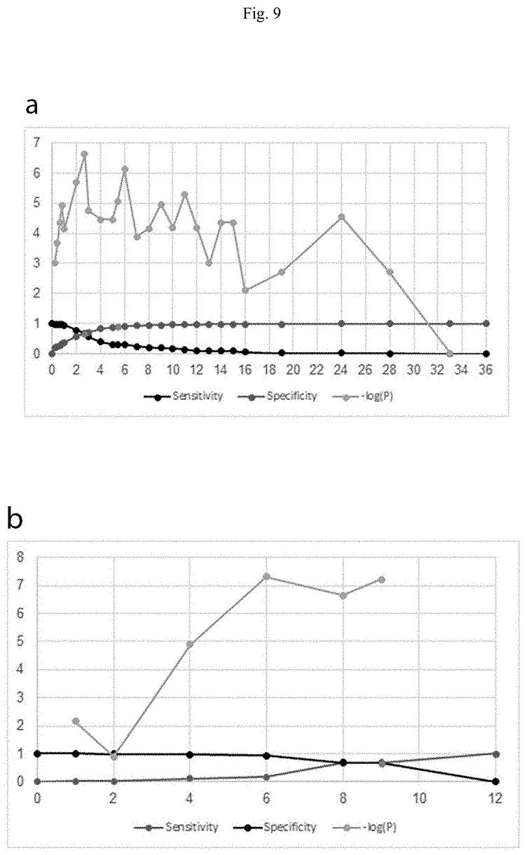

As anticipated, patients with PSA low/Ki67 Q4-high had the worst prognosis (FIG. 8e). Among the GS≤6 patients, PSA low/Ki67 Q4-high were very rare, but it was still obvious that reduced PSA and/or increased Ki67 levels were associated with poor prognosis (FIG. 8f). Notably, the cut-off values for defining PSA/Ki67 high/low should be adjusted with the purpose of increasing sensitivity or specificity, respectively, in relation to the defined application (FIG. 9).

Taken together, those results indicated that a combinatory PSA and Ki67 IR score adds prognostic information to GS in PC patients (Table 4). Furthermore, for identification of patients with a good prognosis a lower Ki67 cut-off level seems superior whereas cases with a particularly poor prognosis are more specifically identified by increasing the Ki67 cut-off value.

### Example 11: Clinical and Histopathological Characteristics of Tumors Categorized by their PSA and Ki67 Immunoreactivity

As the identified subgroups based on PSA and Ki67 staining showed differences in clinical behavior, their characteristics were examined in more detail (using all available cases irrespective of treatment, and the Q4 was used to define high Ki67). The most common group, PSA high/Ki67 Q4-low (141/331, 43% of all cases), contained tumors with an IHC staining pattern similar to that of normal prostate glands, that is homogeneous and intense PSA staining and low cell proliferation. This group was characterized by low GS, low tumor extent and stage, and low fraction of bone metastases at diagnosis (Table 5). Furthermore, they showed low values of various markers in the tumor epithelium and in the tumor stroma previously related to poor outcome in this patient cohort (Tables 5 and 6). Although the PSA high/Ki67 Q4-low subgroup showed the best prognosis, still 18% of the men in this group died from prostate cancer (see below). Using the median Ki67 as cut-off a smaller (106/331) PSA high/Ki67 med-low group where only 12% died from prostate cancer was identified.

The group most different from that above, defined by PSA low/Ki67 Q4-high (68/331, 21% of all cases) was characterized by high GS, high tumor volume and stage, many cases with bone metastases already at diagnosis, and in this group 74% of the patient died from prostate cancer (Tables 5 and 6, FIG. 8). Several markers previously associated with poor outcome showed levels suggesting particularly aggressive disease in this group (Tables 5 and 6). For example, the highest levels of pEGF-R, ErbB2, pAkt, and Erg as a marker for TRMPSS2-ERG fusion gene (38) were found in the tumor epithelium of this group. The tumor stroma showed signs of a reactive response (39, 40) with increased type 2 (CD163+) macrophage infiltration, vascular density and hyaluronic acid, and reduced levels of caveolin-1, androgen receptors and mast cells (Tables 5 and 6). All these tumor characteristics were seen also in the larger group (116/331) defined by PSA low/Ki67 med-high, a group where 66% of the men died from prostate cancer (data not shown).

The 2^{nd} largest group (105/331, 32%) contained cases defined by PSA low/Ki67 Q4-low. Also this group had higher GS, tumor volume, stage, and fraction of cases with bone metastases at diagnosis than the PSA high/Ki67 Q4-low group (Table 5). They also had a less favorable outcome than the PSA high/Ki67 Q4-low group, but the prognosis was better than for the PSA low/Ki67 Q4-high group (Table 5, FIG. 8). Accordingly, markers previously found associated with a poor prognosis suggested that this group scored intermediate between the other groups. About 50% in this group died from prostate cancer (see below).

The group defined by PSA high/Ki67 Q4-high contained very few patients (17/331, 5%) suggesting that the phenotype is uncommon. This group of patients had higher tumor volume and stage and percentage of cases with bone metastases than the group with PSA high/Ki67 low, as well as significantly increased levels of ErbB2 and hyaluronic acid (Table 5).

It was investigated whether the tumor-instructed normal tissue (TINT) response (43) was associated with tumor subtype. Subgroups PSA high/Ki67 Q4-low and PSA low/Ki67 Q4-high, the groups with the best and worst prognosis, respectively, showed some morphological differences in the benign parts of the tumor bearing prostate. The benign parts of prostate carrying PSA low/Ki67 Q4-high tumors was characterized by significantly increased pEGF-R (P<0.01) in the epithelium and increased number of mast cells (P<0.01) in the stroma (Table 4). Epithelial pAkt (P=0.07) and Ki67 (P=0.07) in benign glands, and hyaluronic acid in the stroma (P=0.07) also tended to be increased.

As noted above, disease outcome differed within each subgroup. Patients dying from prostate cancer were compared to those that died from other causes or were alive. In the PSA high/Ki67 Q4-low tumors, the relatively few cases that died from prostate cancer had higher median GS (7 vs. 6, P<0.001), tumor stage; (2 vs. 1, P<0.05), tumor content (60 vs. 10%, P<0.001) and Ki67 index (2.7 vs. 1.2%, P<0.01). They also showed signs of a more pronounced stroma reaction with more hyaluronic acid (8 vs. 7, P<0.05), and blood vessels (14 vs. 11, P<0.05), as well as lower caveolin-1 in the tumor stroma (3 vs. 3, P<0.05) than those alive or dying from other causes.

In the group with PSA low/Ki67 Q4-low where 51% died from prostate cancer, the men who died from prostate cancer had higher GS (8 vs. 6, P<0.001), higher tumor volume (75 vs. 30%, P<0.01), higher stage (3 vs. 1, P<0.001), and more commonly metastases at diagnosis (25 vs. 3%, P<0.01), but their PSA or Ki67 staining scores did not differ from those alive or dying from other causes. They also had higher hyaluronic acid staining in tumor stroma (9 vs. 7, P<0.01), more tumor infiltrating CD163+ macrophages (25 vs. 19, P<0.05), reduced stroma androgen receptors (42 vs. 52, P<0.05) and reduced caveolin-1 (2 vs. 3, P<0.05). The few patients dying from other causes in the PSA low/Ki67 Q4-high group had lower median GS (7 vs. 9, P<0.01) than those dying from prostate cancer. In summary standard prognostic markers like GS and the magnitude of stroma response affected prognosis within the PSA/Ki67 subgroups.

### Example 12: Validation of the MetA-C Subtypes and their Clinical Relevance

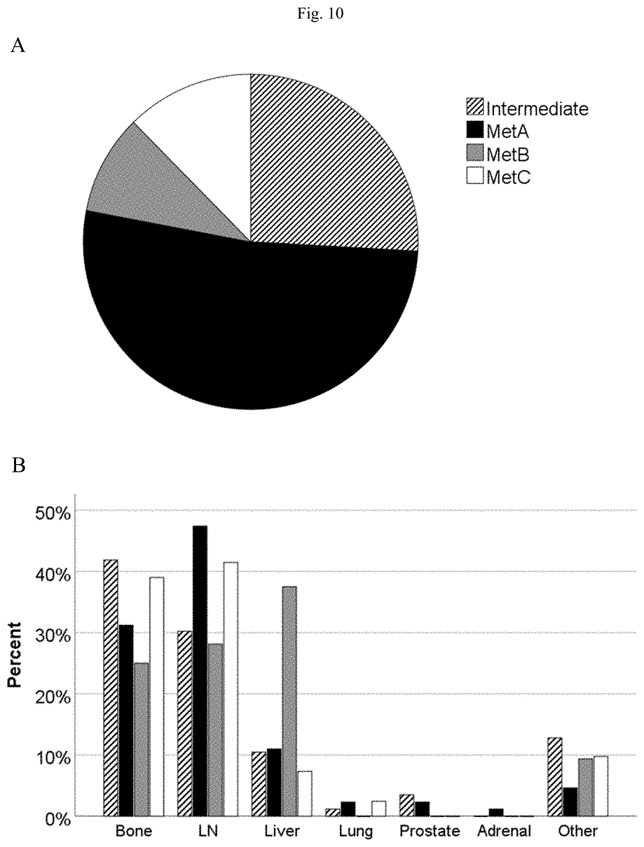

The top 60 differentiating gene transcripts per subtype (Table 1) were used for PCA and OPLS-DA modeling of an external set of PC metastasis (52). Of the original 180 gene products in Table 1, 157 clustered as MetA-C associated transcripts also in the Abida cohort (52) and were further used to build an OPLS-DA classifier for prediction of the molecular subtype in the 332 metastasis cases. The predicted frequency of MetA, MetB, and MetC were 52, 10, and 12%, respectively, while 26% were predicted to have an intermediate class (FIG. 10A). The relatively low MetA and high intermediate frequency in comparison to the original cohort (FIG. 1) might reflect the fact that the patients in Abida cohort were more heavily treated, with a substantial number of patients having received abiraterone or enzalutamide treatment in comparison to none in the original cohort. Interestingly, the MetA-C subtypes were observed at different metastases sites, with the MetB cases being enriched among the liver metastases and the MetA cases among the bone and lymph node metastases (FIG. 10B).

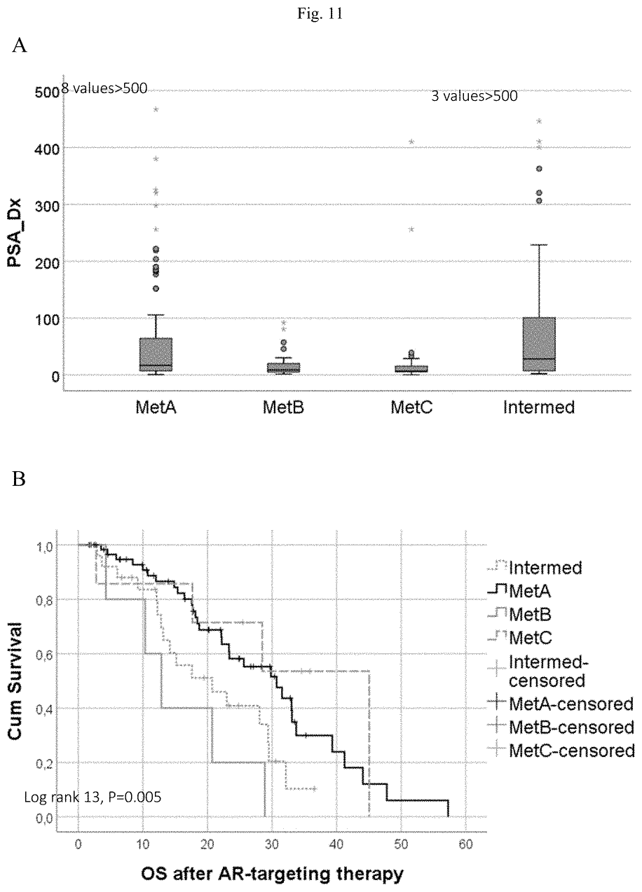

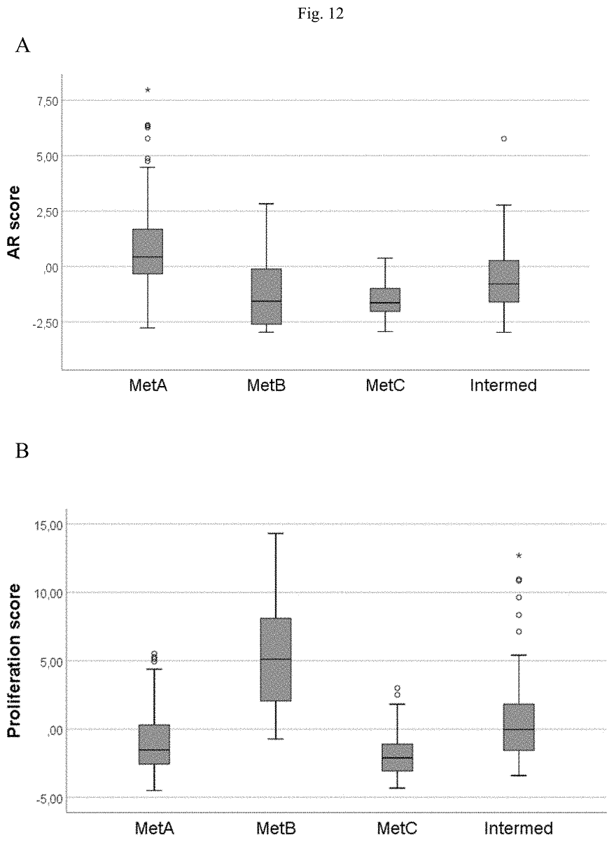

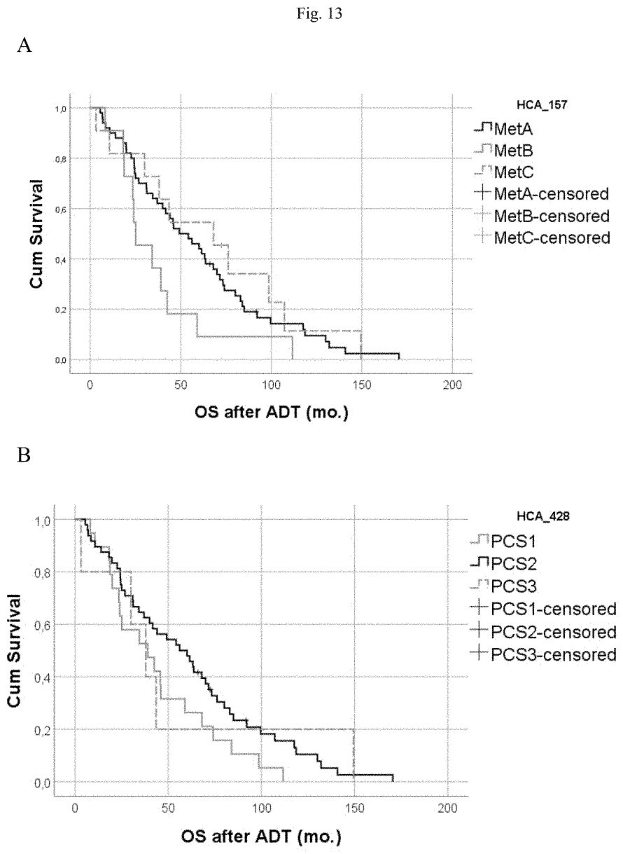

To validate the clinical relevance of the MetA-C subtypes, classes were analyzed in relation to serum PSA levels and survival after androgen receptor targeting therapy given to patients due to castration-resistant disease. As anticipated from the original results, patients with metastases of the MetB and MetC subtypes had lower serum PSA than MetA patients (FIG. 11A) and the MetB patients showed the worst prognosis after AR-targeting therapy (FIG. 11B). Accordingly, MetB and MetC cases had low AR activity, determined from expression levels of AR-regulated genes (defined in (7)), and MetB had high proliferation activity based on expression levels of cell cycle associated genes (defined by the Prolaris test (51)) (FIG. 12).

### Example 13: Performance of the Defined MetA-C-Differentiating Gene Panels in Comparison to Previously Defined PCS1-3-Associated Gene Panels

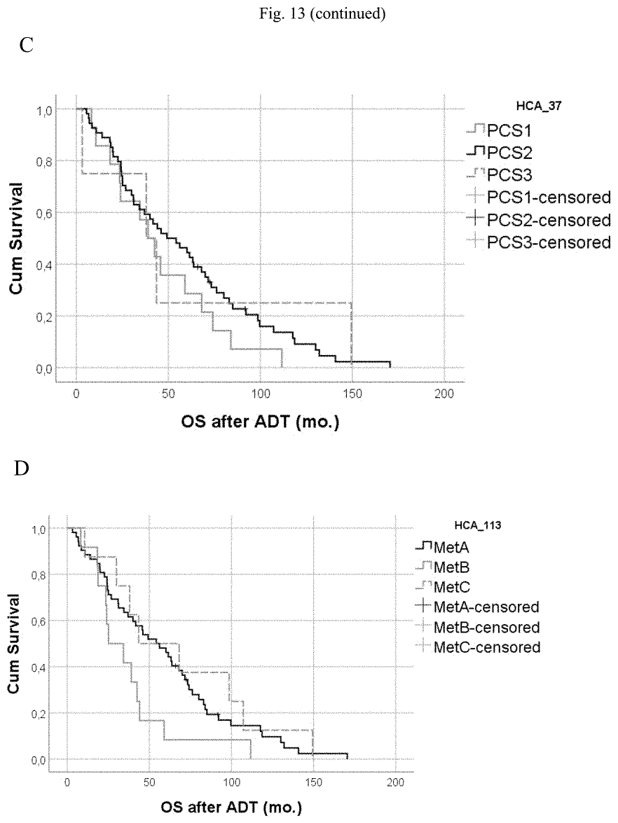

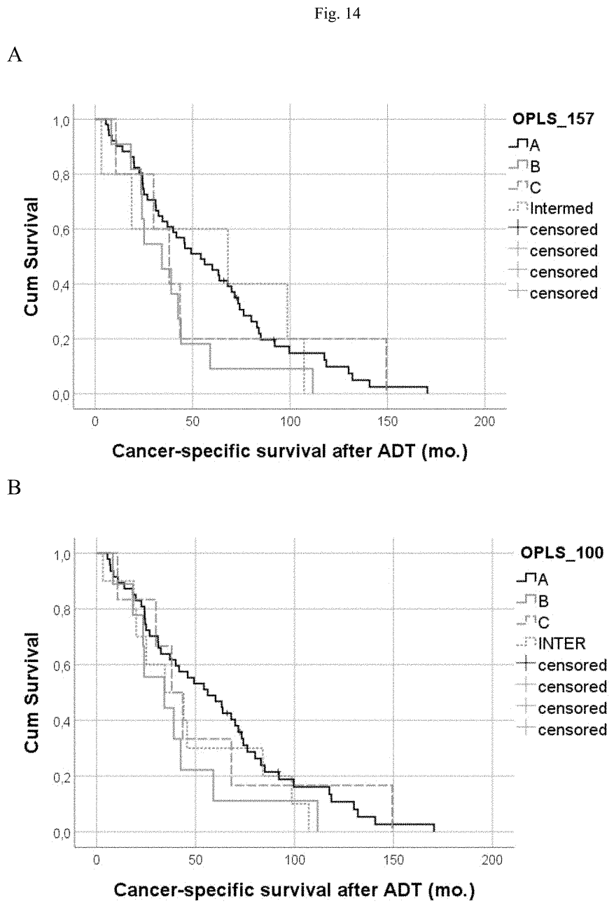

Sets of gene transcripts have been previously defined to differentiate primary prostate tumors into molecular subgroups, PCS1-3, with different prognosis and phenotypes (18; see also WO 2017/062505). The MetA-C subtypes show molecular and phenotypical similarities with the PCS1-3 tumor groups, respectively. Nevertheless, the 157 MetA-C-associated gene panel performed better than the 428 and 37 gene PCS1-3-associated panels suggested by You et al. (18) in unsupervised cluster analysis differentiating of the most aggressive MetB subtype from the rest (FIG. 13). Also, a reduced panel consisting of 113 transcripts with no overlap with the PCS1-3-associated genes performed well in differentiating MetA-C subtypes (FIG. 13B).

### Example 14: Reduced Gene Panels Conserve the Predictive Ability of MetA-C

First, 180 of the gene transcripts shown in Table 1 157 were selected as robustly MetA-C associated, by excluding ACSS1, AP1S2, C9orf91, CTBS, GABARAPL2, LOC399942, LOC642299, LOC643287, LOC730994, LOC731999, LYL1, MDC1, MGC4677, PARVG, PHF16, PSD4, PTMA, PTTG3P, SELT, UBTD1, VPS54, WASF3, ZNF250, based on cluster analysis of the original 72 samples (GSE29650 and GSE101607) and two external data cohorts of PC metastases (50, 52).

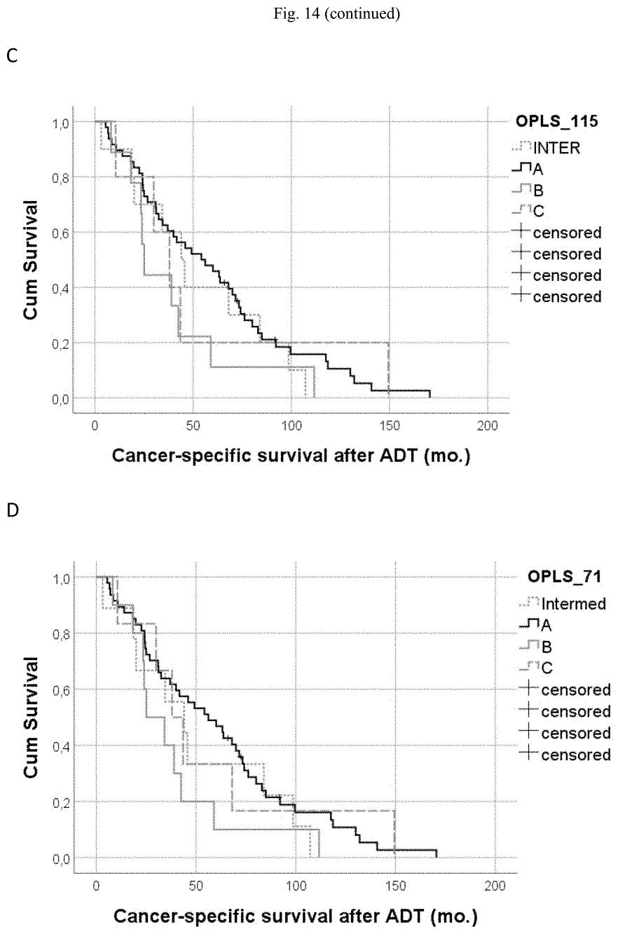

Secondly, the 157-transcript panel was reduced by different means in order to reduce analysis complexity without losing MetA-C predictability: (1) the panel was reduced to 100 transcripts by removing transcripts showing high expression in human lymphocytes (according to the Human Protein Atlas; https://www.proteinatlas.org). Alternatively (2), the panel was reduced to 115 and 71 genes in two steps by (i) excluding transcripts, amidst pairs showing highest similarity by hierarchical cluster analysis, removing the gene with lowest average gene expression intensity in the original 72 samples; and (ii) removing transcripts showing high expression in human lymphocytes (according to the Human Protein Atlas; https://www.proteinatlas.org). The reduced gene panels kept a high predictability for separating MetA-C, and patient prognosis after ADT, as exemplified by the 157-gene, 100-gene, 115-gene and the 71-gene panels (FIG. 14).

The following gene transcripts were identified in the 157-transcript panel:

MetA (50 Genes)
ACAA1, ACP6, ACPP, ALDH1A3, ALDH6A1, ATP2C1, CANT1, CDH1, CDS1, COG3, CPNE4, CRELD1, DHRS7, ENTPD5, ENTPD6, FAM174B, FICD, GREB1, GTF3C1, H2AFJ, HPN, IVD, KIAA0251, KLK2, KLK3, LOC124220, NAAA, NECAB3, NWD1, PLA2G4F, PPAP2A, REXO2, RNF41, SC5DL, SCCPDH, SEC22C, SEC23B, SECISBP2L, SLC25A17, SLC35A3, SLC37A1, SLC39A6, SLC4A4, SLC9A2, SLC9A3R1, STEAP2, SUOX, TSPAN1, VIPR1, XBP1.

MetB (53 Genes)
ASPM, BUB1, C12orf48, C16orf75, C17orf53, C1orf135, C6orf173, CCNA2, CCNB1, CCNB2, CDC2, CDC20, CDC45L, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LSM2, MAD2L1, MCM10, MCM2, MCM7, MEST, MSH6, NCAPG, NUSAP1, OIP5, PSRC1, RACGAP1, RFC5, STIL, STMN1, TOP2A, TPX2, TTK, TUBB, UBE2C, UNG, USP1.

MetC (54 Genes)
AEBP1, ARHGAP23, ARHGEF6, BMP1, C10orf54, C1orf54, C1QTNFS, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KL4A1602, MSN, NAALADL1, NINJ2, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, VAMP5.

The following gene transcripts were identified in the 115-transcript panel:

MetA (38 Genes)
ALDH6A1, C9orf91, CDH1, CPNE4, KL4A0251, KLK2, LOC124220, LOC731999, PPAP2A, REXO2, RNF41, SLC39A6, STEAP2, SUOX, TSPAN1, XBP1, ACAA1, ACP6, ACPP, ACSS1, ALDH1A3, CANT1, CDS1, CRELD1, CTBS, DHRS7, H2AFJ, IVD, KLK3, LOC642299, NAAA, PLA2G4F, SC5DL, SEC22C, SEC23B, SLC25A17, SLC4A4, VPS54.

MetB (39 Genes)
ASPM, C12orf48, C6orf173, KIF15, MCM10, MEST, MSH6, OIP5, STIL, TOP2A, TTK, ZNF250, BUB1, CCNB2, CDC2, CDC20, CDC45L, CDCA4, CKS1B, CKS2, DDX39, DEK, ECT2, FAM83D, GAS2L3, HMGB2, KIF11, KIFC1, LIN9, LOC399942, LOC643287, LSM2, MCM2, NCAPG, NUSAP1, PTMA, RFC5, STMN1, TUBB.

MetC (38 Genes)
BMP1, C1orf54, CD93, CLDN5, COX7A1, DPYSL2, GYPC, ICAM2, JAM3, LYL1, RAB31, SH2B3, STAB1, TCF4, TPM2, TPST2, AEBP1, ARHGAP23, ARHGEF6, C10orf54, CDH5, COL6A3, ENG, FERMT2, FNDC1, FXYD5, GAS6, GIMAP4, KIAA1602, LOC730994, MGC4677, MSN, PDGFRB, PECAM1, RASIP1, STOM, UBTD1, VAMP5.

The following gene transcripts were identified in the 100-transcript panel:

MetA (33 Genes)
VPS54, LOC642299, VIPR1, KLK3, SC5DL, SLC4A4, PLA2G4F, ACP6, SEC23B, H2AFJ, DHRS7, HPN, SLC25A17, CDS1, SEC22C, ACSS1, SLC37A1, CRELD1, SELT, ACPP, GTF3C1, SLC35A3, NAAA, SLC9A3R1, IVD, SLC9A2, GABARAPL2, ENTPD6, CANT1, ACAA1, SECISBP2L, ALDH1A3, CTBS.

MetB (36 Genes)
RFC5, ECT2, DEK, LSM2, GAS2L3, STMN1, MCM7, MDC1, NCAPG, CKS2, LIN9, NUSAP1, CCNB2, TUBB, CDC45L, LOC643287, CDCA4, CDC2, LOC399942, KIFC1, HMGB2, MCM2, PTMA, FAM83D, KIF11, CDC20, KIF20A, CCNB1, CKS1B, DDX39, C1orf135, BUB1, USP1, CENPL, CCNA2, PHF16.

MetC (31 Genes)

MSN, STOM, ITGA5, C10orf54, VAMP5, RASIP1, ENG, COL6A2, CYYR1, MGC4677, SRPX2, PARVG, FAM176B, GAS6, ARHGEF6, PLCG2, LOC730994, PECAM1, COL6A3, GIMAP4, CDH5, FNDC1, KIAA1602, ARHGAP23, UBTD1, SH3KBP1, FERMT2, AEBP1, PDGFRB, AP1S2, FXYD5.

The following gene transcripts were identified in the 71-transcript panel:

MetA (22 Genes)
ACAA1, ACP6, ACPP, ACSS1, ALDH1A3, CANT1, CDS1, CRELD1, CTBS, DHRS7, H2AFJ, IVD, KLK3, LOC642299, NAAA, PLA2G4F, SC5DL, SEC22C, SEC23B, SLC25A17, SLC4A4, VPS54.

MetB (27 Genes)
BUB1, CCNB2, CDC2, CDC20, CDC45L, CDCA4, CKS1B, CKS2, DDX39, DEK, ECT2, FAM83D, GAS2L3, HMGB2, KIF11, KIFC1, LIN9, LOC399942, LOC643287, LSM2, MCM2, NCAPG, NUSAP1, PTMA, RFC5, SIMN1, TUBB.

MetC (22 Genes)
AEBP1, ARHGAP23, ARHGEF6, C10orf54, CDH5, COL6A3, ENG, FERMT2, FNDC1, FXYD5, GAS6, GIMAP4, KIAA1602, LOC730994, MGC4677, MSN, PDGFRB, PECAM1, RASIP1, STOM, UBTD1, VAMP5.

TABLE 1
Top 60 differentiating gene transcripts per subtype.

| MetA-enriched | | MetB-enriched | | MetC-enriched | |
|---|---|---|---|---|---|
| Symbol | Gene ID | Symbol | Gene ID | Symbol | Gene ID |
| ACAA1 | ILMN_1738921 | ASPM | ILMN_1815184 | AEBP1 | ILMN_1736178 |
| ACP6 | ILMN_2234343 | BUB1 | ILMN_2202948 | AP1S2 | ILMN_2120273 |
| ACPP | ILMN_1758323 | C12orf48 | ILMN_1727055 | ARHGAP23 | ILMN_1764571 |
| ACSS1 | ILMN_1752269 | C16orf75 | ILMN_1790537 | ARHGEF6 | ILMN_1803423 |
| ALDH1A3 | ILMN_2139970 | C17orf53 | ILMN_1776490 | BMP1 | ILMN_1800412 |
| ALDH6A1 | ILMN_1785284 | C1orf135 | ILMN_1787280 | C10orf54 | ILMN_2205963 |
| ATP2C1 | ILMN_2340565 | C6orf173 | ILMN_1763907 | C1orf54 | ILMN_1702231 |
| C9orf91 | ILMN_1803652 | CCNA2 | ILMN_1786125 | C1QTNF5 | ILMN_1744487 |
| CANT1 | ILMN_1664012 | CCNB1 | ILMN_1712803 | CAV1 | ILMN_2149226 |
| CDH1 | ILMN_1770940 | CCNB2 | ILMN_1801939 | CD93 | ILMN_1704730 |
| CDS1 | ILMN_1801476 | CDC2 | ILMN_1747911 | CDH5 | ILMN_1719236 |
| COG3 | ILMN_1776154 | CDC20 | ILMN_1663390 | CLDN5 | ILMN_1728197 |
| CPNE4 | ILMN_1814770 | CDC45L | ILMN_1670238 | CLIP3 | ILMN_1789733 |
| CRELD1 | ILMN_1739558 | CDCA3 | ILMN_1737728 | COL6A2 | ILMN_1783909 |
| CTBS | ILMN_2144573 | CDCA4 | ILMN_1684045 | COL6A3 | ILMN_1706643 |
| DHRS7 | ILMN_1807455 | CENPF | ILMN_1664516 | COX7A1 | ILMN_1662419 |
| ENTPD5 | ILMN_1745849 | CENPL | ILMN_1742779 | CYYR1 | ILMN_1812902 |
| ENTPD6 | ILMN_2091792 | CKS1B | ILMN_1719256 | DDR2 | ILMN_2410523 |
| FAM174B | ILMN_1652797 | CKS2 | ILMN_2072296 | DPYSL2 | ILMN_1672503 |
| FICD | ILMN_1778064 | DDX39 | ILMN_1747303 | ENG | ILMN_1760778 |
| GABARAPL2 | ILMN_1796458 | DEK | ILMN_1747630 | FAM176B | ILMN_1769092 |
| GREB1 | ILMN_1721170 | ECT2 | ILMN_1717173 | FERMT2 | ILMN_1695290 |
| GTF3C1 | ILMN_1789839 | FAM83D | ILMN_1781943 | FGD5 | ILMN_2104141 |
| H2AFJ | ILMN_1708728 | GAS2L3 | ILMN_2211003 | FNDC1 | ILMN_1734653 |
| HPN | ILMN_1687235 | HMGB2 | ILMN_1654268 | FXYD5 | ILMN_2309848 |
| IVD | ILMN_1724207 | KIF11 | ILMN_1794539 | GAS6 | ILMN_1779558 |
| KIAA0251 | ILMN_1703969 | KIF15 | ILMN_1753063 | GIMAP4 | ILMN_1748473 |
| KLK2 | ILMN_2371917 | KIF20A | ILMN_1695658 | GIMAP8 | ILMN_1747305 |
| KLK3 | ILMN_1663787 | KIF23 | ILMN_1811472 | GJA4 | ILMN_1671106 |
| LOC124220 | ILMN_1753139 | KIFC1 | ILMN_2222008 | GYPC | ILMN_1668039 |
| LOC642299 | ILMN_1810431 | LIN9 | ILMN_2137084 | ICAM2 | ILMN_1786823 |
| LOC731999 | ILMN_1660277 | LOC399942 | ILMN_1765701 | IGFBP4 | ILMN_1665865 |
| NAAA | ILMN_1668605 | LOC643287 | ILMN_1677906 | ITGA5 | ILMN_1792679 |
| NECAB3 | ILMN_1749738 | LSM2 | ILMN_2070300 | JAM3 | ILMN_1769575 |
| NWD1 | ILMN_1721540 | MAD2L1 | ILMN_1777564 | KIAA1602 | ILMN_1763640 |
| PLA2G4F | ILMN_1744211 | MCM10 | ILMN_2413898 | LOC730994 | ILMN_1680774 |
| PPAP2A | ILMN_2343278 | MCM2 | ILMN_1681503 | LYL1 | ILMN_2216582 |
| PSD4 | ILMN_2154115 | MCM7 | ILMN_1663195 | MGC4677 | ILMN_2143795 |
| REXO2 | ILMN_1749009 | MDC1 | ILMN_1814122 | MSN | ILMN_1659895 |
| RNF41 | ILMN_1808095 | MEST | ILMN_1669479 | NAALADL1 | ILMN_1770963 |
| SC5DL | ILMN_1677607 | MSH6 | ILMN_1729051 | NINJ2 | ILMN_1731745 |
| SCCPDH | ILMN_1795839 | NCAPG | ILMN_1751444 | PARVG | ILMN_1695851 |
| SEC22C | ILMN_2290618 | NUSAP1 | ILMN_1726720 | PDGFRB | ILMN_1815057 |
| SEC23B | ILMN_2366246 | OIP5 | ILMN_2196984 | PECAM1 | ILMN_1689518 |
| SECISBP2L | ILMN_1784333 | PHF16 | ILMN_1790518 | PLCG2 | ILMN_1815719 |
| SELT | ILMN_1746368 | PSRC1 | ILMN_1671843 | PLCL2 | ILMN_1737025 |
| SLC25A17 | ILMN_1737312 | PTMA | ILMN_1759954 | RAB31 | ILMN_1660691 |
| SLC35A3 | ILMN_1653429 | PTTG3P | ILMN_2049021 | RASIP1 | ILMN_1755657 |
| SLC37A1 | ILMN_1687495 | RACGAP1 | ILMN_2077550 | SH2B3 | ILMN_1752046 |
| SLC39A6 | ILMN_1750394 | RFC5 | ILMN_1659364 | SH3KBP1 | ILMN_1810782 |
| SLC4A4 | ILMN_1734897 | STIL | ILMN_2413650 | SLIT3 | ILMN_1811313 |
| SLC9A2 | ILMN_1738849 | STMN1 | ILMN_1657796 | SRPX2 | ILMN_1676213 |
| SLC9A3R1 | ILMN_1680925 | TOP2A | ILMN_1686097 | STAB1 | ILMN_1655987 |
| STEAP2 | ILMN_2344298 | TPX2 | ILMN_1796949 | STOM | ILMN_1766657 |
| SUOX | ILMN_1803745 | TTK | ILMN_1788166 | TCF4 | ILMN_1814194 |
| TSPAN1 | ILMN_1747546 | TUBB | ILMN_2101885 | TEK | ILMN_2066151 |
| WASF3 | ILMN_1810797 | UBE2C | ILMN_2301083 | TPM2 | ILMN_1789196 |
| VIPR1 | ILMN_2199389 | UNG | ILMN_1683120 | TPST2 | ILMN_2329679 |
| VPS54 | ILMN_1761086 | USP1 | ILMN_1696975 | UBTD1 | ILMN_1794914 |
| XBP1 | ILMN_1809433 | ZNF250 | ILMN_1757230 | VAMP5 | ILMN_1809467 |

The term “Gene ID” refers to the Illumina BeadChips microarray probe accession number in the NCBI Probe database (www.ncbi.nlm.nih.gov/probe).

TABLE 2
Patient characteristics at prostate cancer diagnosis and at time for metastasis surgery in relation to
metastasis subtypes MetA-C^{a}.

| | MetA^{a} | | MetB^{a} | | MetC^{a} | |
|---|---|---|---|---|---|---|
| | n = 51 | | n = 12 | | n = 9 | |
| Age diagnosis (yrs) | 71 | (66; 76) | 64 | (59; 76) | 71 | (63; 76) |
| Age metastasis surgery (yrs) | 74 | (69; 80) | 68 | (62; 76)^{P = 0.084} | 74 | (71; 79) |
| PSA diagnosis (ng/ml) | 160 | (58; 920) | 45 | (19; 76)* | 81 | (29; 130)^{P = 0.075} |
| PSA metastasis surgery (ng/ml) | 470 | (110; 1100) | 84 | (44; 330)^{P = 0.059} | 120 | (110; 180)^{P = 0.068} |
| Follow-up from diagnosis (mo.) | 56 | (29; 84) | 30 | (24; 65) | 43 | (30; 110) |
| Follow up from first ADT^{b} | 54 | (25; 78) | 30 | (21; 43) | 43 | (30; 98) |
| (mo.) | | | | | | |
| Follow up from metastasis | 10 | (3; 33) | 5 | (2; 11) | 13 | (5; 19) |
| surgery (mo.) | | | | | | |
| Gleason score at diagnosis | | | | | | |
| 7 | 13 | (25%) | 3 | (25%) | 3 | (33%) |
| 8 | 13 | (25%) | 2 | (17%) | 4 | (44%) |
| 9 | 10 | (20%) | 3 | (25%) | 1 | (11%) |
| Not available | 15 | (29%) | 4 | (33%) | 1 | (11%) |
| Treatment with curative intention | | | | | | |
| Radical prostatectomy | 1 | (2%) | 0 | (0%) | 1 | (11%) |
| Radiation | 3 | (6%) | 4 | (33%)** | 1 | (11%) |
| Previous ADT^{b}: | | | | | | |
| None | 9 | (18%) | 1 | (8%) | 2 | (22%) |
| Short-tem^{c} | 4 | (8%) | 0 | (0%) | 0 | (0%) |
| Long-term | 38 | (74%) | 11 | (92%) | 7 | (78%) |
| Additional therapies: | | | | | | |
| Bicalutamide | 17 | (33%) | 8 | (67%)* | 5 | (56%) |
| Chemotherapy | 4 | (8%) | 4 | (33%)* | 1 | (11%) |
| Ra223 | 3 | (6%) | 1 | (8%) | 1 | (11%) |
| Bisphosphonate | 5 | (10%) | 1 | (8%) | 1 | (11%) |
| Radiation towards operation site | 7 | (14%) | 1 | (8%) | 1 | (11%) |
| Soft tissue metastases | 9 | (18%) | 5 | (42%)^{P = 0.072} | 1 | (11%)^{P = 0.053} |
| Cancer cells^{d} (%) | 70 | (60; 80) | 70 | (70; 80) | 50 | (35; 50)** |

Continuous variables given as median (25th; 75th percentiles),
*P < 0.05;
**P < 0.01, compared to MetA.
^{a}Metastasis subtype, MetA-C, as determined from principal component analysis of whole genome expression profiles followed by unsupervised clustering (see materials and methods for details)
^{b}Androgen deprivation therapy (ADT) included surgical ablation or LHRH/GnRH agonist therapy.
^{c}ADT for 2-17 days before metastasis surgery.
^{d}Fraction of cancer cell content in frozen metastasis sections extracted for RNA and analyzed by whole genome expression analysis.

TABLE 3
Molecular metastasis subtypes MetA-C^{a} in relation to metastasis and primary tumor morphology.

| | | MetA | | MetB | | MetC | |
|---|---|---|---|---|---|---|---|
| | | (n = 51) | | (n = 12) | | (n = 9) | |
| Bone | AR score (0-12) | 8 | (4; 12) | 10 | (6; 12) | 9 | (4; 12) |
| metastases | | (n = 49) | | (n = 12) | | (n = 8) | |
| | PSA score (0-12) | 9 | (6; 12) | 2 | (1; 6)***a | 6 | (1; 9)a* |
| | | (n = 51) | | (n = 12) | | (n = 9) | |
| | Ki67 (%) | 14 | (9; 20) | 33 | (22; 45)***a | 12 | (8; 28)b* |
| | | (n = 50) | | (n = 12) | | (n = 9) | |
| | Chromogranin A (%) | 0 | (0; 0.2) | 0.2 | (0; 1.6)^{P = 0.05}a | 0 | (0; 0) |
| | | (n = 45) | | (n = 12) | | (n = 9) | |
| | Cellular atypia | 34; 17 | | 2; 10**a | | 2; 7*a | |
| | (moderate; high) | | | | | | |
| | Gland formation | 20; 31 | | 0; 12**a | | 4; 5*a | |
| | (yes; no) | | | | | | |
| | | MetA | | MetB | | MetC | |
| | | associated | | associated | | associated | |
| | | (n = 36) | | (n = 8) | | (n = 8) | |
| Primary | AR score (0-12) | 12 | (12; 12) | 12 | (10; 12) | 10.5 | (8; 12) |
| tumor | | (n = 34) | | (n = 8) | | (n = 8) | |
| | PSA score (0-12) | 9 | (8; 12) | 6 | (4; 8)a** | 7 | (6; 10.5) |
| | | (n = 32) | | (n = 8) | | (n = 8) | |
| | Ki67 (%) | 9 | (6.5; 14) | 19 | (15; 26)a** | 17 | (11; 30)a* |
| | | (n = 35) | | (n = 8) | | (n = 8) | |
| | AR tumor stroma score | 22 | (15; 30) | 11 | (4; 17)a* | 17 | (7; 20) |

| | | | |
|---|---|---|---|
| (% of stroma cells | (n = 34) | (n = 8) | (n = 8) |
| positive) | | | |
| Reactive stroma score | 11; 11; 0 | 0; 2; 7***a | 0; 6; 1*a, b* |
| (1; 2; 3) | | | |

Continuous variables given as median (25th; 75th percentiles)
*P < 0.05,
***P < 0.00,
a = significantly different from MetA,
b = significantly different from MetB
^{a}Metastasis subtype, MetA-C, as determined from principal component analysis of whole genome expression profiles followed by unsupervised clustering (see FIG. 1)

TABLE 4
Multivariate Cox analysis of PSA and Ki67 immunoreactivity
and Gleason score (GS) in relation to cancer-specific
survival diagnosed of patients at TUR-P and managed
by watchful-waiting.

| | | 95% CI | | |
|---|---|---|---|---|
| | HR | Lower | Upper | P |
| GS ≤ 6, n = 131 | 1 | | | |
| GS = 7, n = 47 | 3.8 | 1.8 | 8.1 | 4.8E−04 |
| GS ≥ 8, n = 59 | 6.7 | 3.2 | 14 | 4.9E−07 |
| PSA IR = 12, n = 132 | 1 | | | |
| PSA IR < 12, n = 105 | 2.1 | 1.1 | 3.7 | 0.017 |
| Ki67 (%) | 1.05 | 1.0 | 1.1 | 0.038 |

PSA immunoreactivity (IR) was dichotomized by the median value 9 as high (IR = 12) or low (≤9).
Fraction of Ki67 positive tumor cells was analyzed as a continuous variable.
CI = confidence interval.

TABLE 5
Clinical and histopathological variables in patients stratified by
differences in Ki67 and PSA immunostaining.

| | PSAhigh/ | | PSAhigh/ | | PSAlow/ | | PSAlow/ | |
|---|---|---|---|---|---|---|---|---|
| Clinical | Ki67low | | Ki67high | | Ki67low | | Ki67high | |
| markers | (n = 141, 42%) | | (n = 17, 5%) | | (n = 105, 32%) | | (n = 68, 20%) | |
| Age | 74 | (69; 78) | 75 | (71; 79) | 74 | (69; 78)***a | 75 | (69; 82)***a, ***b |
| GS | | | | | | | | |
| 4-6 | 95 | (67) | 9 | (53) | 37 | (35) | 7 | (10) |
| 7 | 29 | (21) | 3 | (18) | 23 | (22) | 7 | (10) |
| 8-10 | 17 | (12) | 5 | (29)*a | 45 | (43)***a | 54 | (79)***a, **b |
| Tumor stage | | | | | | | | |
| T1 | 94 | (67) | 7 | (41) | 38 | (36) | 11 | (16) |
| T2 | 35 | (25) | 5 | (29) | 31 | (30) | 20 | (29) |
| T3-4 | 11 | (7.8) | 5 | (29) | 33 | (31) | 34 | (50) |
| x | 1 | (0.7) | 0*a | | 3 | (3)**a | 3 | (4)***a, **b |
| M stage | | | | | | | | |
| 0 | 100 | (71) | 11 | (65) | 73 | (70) | 35 | (51) |
| 1 | 3 | (2) | 2 | (12) | 13 | (12) | 23 | (34) |
| x | 38 | (27) | 4 | (24) | 19 | (18) | 10 | (15) |
| Cancer (%) | 10 | (7.5; 45) | 30 | (10; 70) | 60 | (20; 85)***a | 88 | (50; 95)***a, ***b |
| PC death (%) | 26 | (18) | 5 | (29) | 51 | (49)***a | 50 | (74)***a, **b |
| Tumor markers | | | | | | | | |
| pEGF-R | 3.1 | (2.4; 3.6) | 3.6 | (3.1; 3.9) | 3.3 | (2.8; 3.6) | 3.6 | (3.3; 4.0)***a, **b |
| score (36) | (n = 110) | | (n = 8) | | (n = 83) | | (n = 45) | |
| (epithelial) | | | | | | | | |
| ErbB2 | 2.8 | (2.0; 3.0) | 3.0 | (2.7; 3.8)**a | 3.0 | (2.3; 3.8)**a | 3.0 | (3.0; 4.0)***a, *b |
| score (42) | (n = 126) | | (n = 14) | | (n = 99) | | (n = 63) | |
| (epithelial) | | | | | | | | |
| ERG (43) | 105 | (79.5) | 10 | (62.5) | 43 | (44.3) | 21 | (32.3) |
| negative | | | | | | | | |
| positive | 27 | (20.5) | 6 | (37.5) | 54 | (55.7)***a | 44 | (67.7) ***a |
| (epithelial) | | | | | | | | |
| pAkt | 2.6 | (2.2; 2.9) | 2.8 | (2.5; 3.3) | 2.8 | (2.4; 3.1)**a | 3.1 | (2.8; 3.6)***a, ***b |
| score (44) | (n = 109) | | (n = 12) | | (n = 81) | | (n = 49) | |
| (epithelial) | | | | | | | | |
| Ki67 (%) | 1.4 | (0.4; 2.7) | 8.8 | (7.5; 13.6)***a, ***b | 2.5 | (1.2; 3.6)***a | 10.9 | (7.2; 15.6)***a, ***b |
| (11, 12) | (n = 141) | | (n = 17) | | (n = 105) | | (n = 68) | |
| (epithelial) | | | | | | | | |
| Vascular | 11 | (8; 16) | 16 | (9; 19) | 15 | (10; 21)**a | 19 | (12; 24)***a, *b |
| density (%) | (n = 138) | | (n = 17) | | (n = 101) | | (n = 68) | |
| (11, 12) | | | | | | | | |
| Hyaluronic | 7.1 | (4.6; 9.0) | 9 | (6; 11)*a | 7.8 | (5.6; 9.8)*a | 8.6 | (6.2; 11.3)***a |
| acid score | (n = 139) | | (n = 17) | | (n = 105) | | (n = 67) | |
| (45) (stroma) | | | | | | | | |
| Mast cell | 13 | (9; 16) | 14 | (7; 17) | 12 | (8; 16) | 8 | (4; 13)***a, ***b |
| density (%) | (n = 134) | | (n = 16) | | (n = 100) | | (n = 65) | |
| (48) | | | | | | | | |
| Androgen | 50 | (39; 65) | 52 | (22; 67) | 48 | (28; 64) | 37 | (14; 55)***a, **b |
| receptor (%) | (n = 136) | | (n = 16) | | (n = 103) | | (n = 67) | |
| (15) (stroma) | | | | | | | | |
| Caveolin-1 | 3.0 | (2.8; 3.4) | 3.1 | (2.9, 3.4) | 3.0 | (2.8; 3.3)*a | 2.8 | (2.6; 3.1)***a, **b |
| score (46) | (n = 139) | | (n = 16) | | (n = 101) | | (n = 64) | |
| (stroma) | | | | | | | | |
| CD163 (%) | 16 | (11; 22) | 21 | (12; 30) | 19 | (16; 28)***a | 19 | (14; 26) |
| (47) | (n = 87) | | (n = 4) | | (n = 53) | | (n = 29) | |
| TINT markers | | | | | | | | |
| Ki67 (%) | 0.2 | (0; 1.2) | 0 | (0; 1.3) | 0.3 | (0; 1.2) | 0.5 | (0; 2.5) |
| (11, 12) | (n = 138) | | (n = 17) | | (n = 95) | | (n = 57) | |
| (epithelial) | | | | | | | | |
| pEGF-R | 3.0 | (1.8; 3.5) | 2.7 | (2.1; 3.5) | 3.3 | (2.5; 3.8)**a | 3.5 | (3; 3.9) **a |
| score (36) | (n = 111) | | (n = 9) | | (n = 79) | | (n = 40) | |
| (epithelial) | | | | | | | | |
| pAKT | 2.0 | (1.5; 2.5) | 2.0 | (1.4; 2.3) | 2.3 | (1.6; 2.8) | 2.4 | (1.5; 2.8) |
| score (44) | (n = 92) | | (n = 11) | | (n = 56) | | (n = 34) | |
| (epithelial) | | | | | | | | |
| ERG (43) | | | | | | | | |
| negative | 117 | (92.9) | 13 | (76.5) | 75 | (85.2) | 40 | (83.3) |
| positive | 9 | (7.1) | 4 | (23.5)*a | 13 | (14.8) | 8 | (16.7) *a |
| (epithelial) | | | | | | | | |
| Hyaluronic | 6.3 | (4.3; 8.0) | 5.5 | (3.8; 8.1) | 6.5 | (5.0; 9.0) | 7 | (5; 9) |
| acid score | (n = 135) | | (n = 17) | | (n = 99) | | (n = 59) | |
| (45) (stroma) | | | | | | | | |
| Mast cell | 12 | (8; 15) | 12 | (9; 16) | 14 | (10; 20)**a | 14 | (11; 20) **a |
| density (%) | n = 130 | | (n = 17) | | (n = 91) | | (n = 53) | |
| (48) | | | | | | | | |

Continuous variables given as median (25th; 75th percentiles). Ordinal variables given as number (percentage).
x = unknown
a = significantly different from PSA high/Ki67 low
b = significantly different from PSA low/Ki67 low
Mann Whitney U test or Chi square test,
*p < 0.05,
**p < 0.01,
***p < 0.001

TABLE 6
Significant Spearman rank correlations between tumor PSA score and
Ki67 labeling index with other previously measured variables of prognostic
significance (see Table 5 for references) describing tumor and surrounding normal
prostate tissue (TINT).

| | | | Correlation coefficient | |
|---|---|---|---|---|
| | Correlation coefficient | | for tumor Ki67 labeling | |
| | for tumor PSA score | | index | |
| Clinical markers | | | | |
| Gleason score | −0.54*** | (n = 346) | 0.50*** | (n = 389) |
| Tumor stage | −0.41*** | (n = 339) | 0.42*** | (n = 382) |
| M stage | −0.31*** | (n = 272) | 0.33*** | (n = 301) |
| Cancer (%) | −0.47*** | (n = 346) | 0.45*** | (n = 389) |
| Overall survival | 0.21*** | (n = 346) | −0.15** | (n = 389) |
| Tumor markers | | | | |
| Ki67 (%) | −0.46*** | (n = 331) | | |
| pEGF-R score | −0.21** | (n = 252) | 0.28*** | (n = 293) |
| pAkt score | −0.31*** | (n = 255) | 0.36*** | (n = 278) |
| ErbB2 score | −0.29*** | (n = 307) | 0.29*** | (n = 350) |
| Vascular density (%) | −0.24*** | (n = 330) | 0.28*** | (n = 381) |
| Hyaluronic acid score (stroma) | −0.18** | (n = 334) | 0.27*** | (n = 384) |
| Mast cell density (%) | 0.21*** | (n = 322) | −0.13* | (n = 362) |
| Androgen receptor (%) (stroma) | 0.17** | (n = 329) | −0.17** | (n = 373) |
| PDGFR-beta (stroma) (37) | −0.15* | (n = 248) | 0.21*** | (n = 283) |
| Caveolin-1 score (stroma) | 0.25*** | (n = 326) | −0.25*** | (n = 370) |
| CD163 (%) | −0.24** | (n = 177) | | |
| Erg (positive or not) | −0.39*** | (n = 315) | 0.32*** | (n = 350) |
| TINT markers | | | | |
| Ki67 | | | 0.17** | (n = 360) |
| pEGF-R | −0.19** | (n = 244) | 0.25*** | (n = 284) |
| PDGFR-beta (stroma) (37) | −0.13* | (n = 302) | 0.15** | (n = 344) |
| Hyaluronic acid score (stroma) | −0.14* | (n = 318) | 0.14** | (n = 363) |
| Mast cell density (%) | −0.22*** | (n = 299) | | |
| Caveolin-1 score (stroma) | | | −0.11* | (n = 352) |
| Erg (positive or not) | | | 0.16** | (n = 331) |

*Correlation is significant at the < 0.05 level (2-tailed)
**Correlation is significant at the < 0.01 level (2-tailed)
***Correlation is significant at the < 0.001 level (2-tailed)

### REFERENCES

1. Omlin A, Pezaro C, Gillessen Sommer S. Sequential use of novel therapeutics in advanced prostate cancer following docetaxel chemotherapy. Ther Adv Urol 2014; 6:3-14.
2. Van Etten J L, Dehm S M. Clonal origin and spread of metastatic prostate cancer. Endocr Relat Cancer. 2016; 23:R207-17.
3. Bergström S H, Rudolfsson S H, Bergh A. Rat Prostate Tumor Cells Progress in the Bone Microenvironment to a Highly Aggressive Phenotype. Neoplasia 2016; 18:152-61.
4. Hornberg E, Ylitalo E B, Crnalic S, et al. Expression of androgen receptor splice variants in prostate cancer bone metastases is associated with castration-resistance and short survival. PLoS One 2011; 6:e19059.
5. Jernberg E, Thysell E, Bovinder Ylitalo E, et al. Characterization of prostate cancer bone metastases according to expression levels of steroidogenic enzymes and androgen receptor splice variants. PLoS One 2013; 8:e77407.
6. Djusberg E, Jernberg E, Thysell E, et al. High levels of the AR-V7 Splice Variant and CoAmplification of the Golgi Protein Coding YIPF6 in AR Amplified Prostate Cancer Bone Metastases. Prostate 2017; 77:625-38.
7. Ylitalo E B, Thysell E, Jernberg E, et al. Subgroups of Castration-resistant Prostate Cancer Bone Metastases Defined Through an Inverse Relationship Between Androgen Receptor Activity and Immune Response. Eur Urol 2017; 71:776-87.7.
8. Nordstrand A, Bovinder Ylitalo E, Thysell E, et al. Bone Cell Activity in Clinical Prostate Cancer Bone Metastasis and Its Inverse Relation to Tumor Cell Androgen Receptor Activity. Int J Mol Sci 2018; 19.
9. Iglesias-Gato D, Thysell E, Tyanova S, Crnalic S, Santos A., Lima T S, Geiger T., Cox J, Widmark A, Bergh A, Mann M, Flores-Morales A., Wikström P. The proteome of prostate cancer bone metastasis reveals phenotypic heterogeneity with prognostic and therapeutic implications. Clin Cancer Res 2018 Jul. 24. pii: clincanres.1229.2018. doi: 10.1158/1078-0432.CCR-18-1229. [Epub ahead of print]
10. Crnalic S, Hornberg E, Wikström P, et al. Nuclear androgen receptor staining in bone metastases is related to a poor outcome in prostate cancer patients. Endocr Relat Cancer 2010; 17:885-95.
11. Josefsson A, Wikstrom P, Egevad L, et al. Low endoglin vascular density and Ki67 index in Gleason score 6 tumours may identify prostate cancer patients suitable for surveillance. Scand J Urol 2012; 46:247-57.
12. Josefsson A, Wikstrom P, Granfors T, et al. Tumor size, vascular density and proliferation as prognostic markers in GS 6 and GS 7 prostate tumors in patients with long follow-up and non-curative treatment. Eur Urol 2005; 48:577-83.
13. Frey B J, Dueck D. Clustering by passing messages between data points. Science 2007; 315:972-6.
14. Halin S, Hammarsten P, Wikström P, et al. Androgen-insensitive prostate cancer cells transiently respond to castration treatment when growing in an androgen-dependent prostate environment. Prostate 2007; 67:370-7.
15. Wikstrom P, Marusic J, Stattin P, et al. Low stroma androgen receptor level in normal and tumor prostate tissue is related to poor outcome in prostate cancer patients. Prostate 2009; 69:799-809.
16. Saeter T, Vlatkovic L, Waaler G, et al. The prognostic value of reactive stroma on prostate needle biopsy: a population-based study. Prostate 2015; 75:662-71.
17. Wierstra I, Alves J. FOXM1, a typical proliferation-associated transcription factor. Biol Chem 2007; 388:1257-74.
18. You S, Knudsen B S, Erho N, et al. Integrated Classification of Prostate Cancer Reveals a Novel Luminal Subtype with Poor Outcome. Cancer Res 2016; 76:4948-58.
19. Zhao S G, Chang S L, Erho N, et al. Associations of Luminal and Basal Subtyping of Prostate Cancer With Prognosis and Response to Androgen Deprivation Therapy. JAMA Oncol 2017; 3:1663-72.
20. Flores-Morales A, Bergmann T B, Lavallee C, et al. Proteogenomic characterization of patient-derived xenografts highlights the role of REST in neuroendocrine differentiation of castration-resistant prostate cancer. Clin Cancer Res. 2018 Oct. 1. pii: clincanres.0729.2018. doi:10.1158/1078-0432.CCR-18-0729. [Epub ahead of print].
21. Cai C, He H H, Chen S, et al. Androgen receptor gene expression in prostate cancer is directly suppressed by the androgen receptor through recruitment of lysine-specific demethylase 1. Cancer cell 2011;20:457-71.
22. Gao S, Gao Y, He H H, et al. Androgen Receptor Tumor Suppressor Function Is Mediated by Recruitment of Retinoblastoma Protein. Cell Rep 2016; 17:966-76.
23. Yang S, Jiang M, Grabowska M M, et al. Androgen receptor differentially regulates the proliferation of prostatic epithelial cells in vitro and in vivo. Oncotarget 2016; 7:70404-19.
24. Sharma N L, Massie C E, Ramos-Montoya A, et al. The androgen receptor induces a distinct transcriptional program in castration-resistant prostate cancer in man. Cancer cell 2013;23:35-47.
25. Liu S, Kumari S, Hu Q, et al. A comprehensive analysis of coregulator recruitment, androgen receptor function and gene expression in prostate cancer. eLife 2017; 6.
26. Gao N, Zhang J, Rao M A, et al. The role of hepatocyte nuclear factor-3 alpha (Forkhead Box A1) and androgen receptor in transcriptional regulation of prostatic genes. Mol Endocrinol 2003; 17:1484-507.
27. Ketola K, Munuganti RSN, Davies A, et al. Targeting Prostate Cancer Subtype 1 by Forkhead Box M1 Pathway Inhibition. Clin Cancer Res 2017; 23:6923-33.
28. Horning A M, Wang Y, Lin C K, et al. Single-Cell RNA-seq Reveals a Subpopulation of Prostate Cancer Cells with Enhanced Cell-Cycle-Related Transcription and Attenuated Androgen Response. Cancer Res 2018; 78:853-64.28. Cheville J C, Tindall D, Boelter C, et al. Metastatic prostate carcinoma to bone: clinical and pathologic features associated with cancer-specific survival. Cancer 2002; 95:1028-36.
29. Barakat D J, Zhang J, Barberi T, et al. CCAAT/Enhancer binding protein beta controls androgen-deprivation-induced senescence in prostate cancer cells. Oncogene 2015; 34:5912-22.
30. Duffy M J, Harbeck N, Nap M, et al. Clinical use of biomarkers in breast cancer: Updated guidelines from the European Group on Tumor Markers (EGTM). Eur J Cancer 2017;75:284-98.
31. Bubendorf L, Tapia C, Gasser TC, et al. Ki67 labeling index in core needle biopsies independently predicts tumor-specific survival in prostate cancer. Hum Pathol 1998; 29:949-54.
32. Stege R, Grande M, Carlstrom K, et al. Prognostic significance of tissue prostate-specific antigen in endocrine-treated prostate carcinomas. Clin Cancer Res 2000; 6:160-5.
33. Fisher G, Yang ZH, Kudahetti S, et al. Prognostic value of Ki-67 for prostate cancer death in a conservatively managed cohort. Br J Cancer 2013; 108:271-7.
34. Brechbuhl H M, Finlay-Schultz J, Yamamoto T M, et al. Fibroblast Subtypes Regulate Responsiveness of Luminal Breast Cancer to Estrogen. Clin Cancer Res 2017; 23:1710-21.
35. Roswall P, Bocci M, Bartoschek M, et al. Microenvironmental control of breast cancer subtype elicited through paracrine platelet-derived growth factor-CC signaling. Nature Med 2018; 24:463-73.
36. Hammarsten P, Karalija A, Josefsson A, et al. Low levels of phosphorylated epidermal growth factor receptor in nonmalignant and malignant prostate tissue predict favorable outcome in prostate cancer patients. Clin Cancer Res 2010; 16:1245-55.
37. Egevad L, Granfors T, Karlberg L,et al. Prognostic value of the Gleason score in prostate cancer. BJU Int. 2002; 89:538-42.
38. van Leenders G J, Boormans J L, Vissers C J, et al. Antibody EPR3864 is specific for ERG genomic fusions in prostate cancer: implications for pathological practice. Mod Pathol 2011; 24:1128-38.
39. Barron D A, Rowley D R. The reactive stroma microenvironment and prostate cancer progression. Endocr Relat Cancer 2012; 19:R187-204.
40. Hagglof C, Bergh A. The stroma-a key regulator in prostate function and malignancy. Cancers 2012; 4:531-48.
41. Adamo H H, Strömvall K, Nilsson M, et al. Adaptive (TINT) Changes in the Tumor Bearing Organ Are Related to Prostate Tumor Size and Aggressiveness. PLoS One 2015; 10:e0141601.
42. Hammarsten P, Winther J, Rudolfsson S H, et al. ErbB2 receptor immunoreactivity in prostate cancer: relationship to the androgen receptor, disease severity at diagnosis and disease outcome. PLoS One 2014; 9:e105063.
43. Hagglöf C, Hammarsten P, Strömvall K, et al. TMPRSS2-ERG expression predicts prostate cancer survival and associates with stromal biomarkers. PLoS One 2014; 9:e86824.35.
44. Hammarsten P, Cipriano M, Josefsson A, et al. Phospho-Akt immunoreactivity in prostate cancer: relationship to disease severity and outcome, Ki67 and phosphorylated EGFR expression. PLoS One 2012; 7:e47994.
45. Josefsson A, Adamo H, Hammarsten P, et al. Prostate cancer increases hyaluronan in surrounding nonmalignant stroma, and this response is associated with tumor growth and an unfavorable outcome. Am J Pathol 2011; 179:1961-8.
46. Hammarsten P, Dahl Scherdin T, Hägglöf C, et al. High Caveolin-1 Expression in Tumor Stroma Is Associated with a Favourable Outcome in Prostate Cancer Patients Managed by Watchful Waiting. PLoS One 2016; 11:e0164016.
47. Lundholm M, Hagglof C, Wikberg M L, et al. Secreted Factors from Colorectal and Prostate Cancer Cells Skew the Immune Response in Opposite Directions. Sci Rep 2015; 5:15651.
48. Johansson A, Rudolfsson S, Hammarsten P, et al. Mast cells are novel independent prognostic markers in prostate cancer and represent a target for therapy. Am J Pathol 2010; 177:1031-41.
49. Bylesjo, M., et al. OPLS discriminant analysis: combining the strengths of PLS-DA and SIMCA classification. J Chemometr 2006; 20:341-351.
50. Quigley D A, Dang H X, Zhao S G, et al. Genomic Hallmarks and Structural Variation in Metastatic Prostate Cancer. Cell. 2018; 175:889.
51. Loeb S, Ross A E. Genomic testing for localized prostate cancer: where do we go from here? Curr Opin Urol. 2017 September; 27(5):495-499.
52. Abida W, Cyrta J, Heller G, et al. Genomic correlates of clinical outcome in advanced prostate cancer. PNAS 2019; 116(23):11428-11436.

1. A diagnostic method for classifying a prostate cancer subtype in a sample, said sample comprising tumor-derived material from a subject diagnosed with prostate cancer, said method comprising:
(a) obtaining a gene expression profile from the sample;
(b) comparing the obtained gene expression profile with a reference gene expression subtype profile selected from:
(i) subtype MetA, characterized by increased expression compared to MetB and MetC, of at least 10 of the genes selected from the group consisting of ACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC3SA3, SLC37A1, SLC39A6, SLC4A4, SLC9A2, SLC9A3R1, STEAP2, SUOX, TSPAN1, WASF3, VIPR1, VPSS4, and XBP1;
(ii) subtype MetB, characterized by increased expression compared to MetA and MetC, of at least 10 of the genes selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKS1B, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USPJ, and ZNF250; and
(iii) subtype MetC, characterized by increased expression compared to MetA and MetB, of at least 10 of the genes selected from the group consisting of AEBP1, AP1S2, ARHGAP23, ARHGEF6, BMPJ, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5;
(c) on basis of similarity found in the comparison, classifying the sample as prostate cancer subtype MetA, MetB or MetC.

2. The method according to claim 1 wherein the reference gene expression profiles are obtained from bone metastases tissue from prostate cancer patients.

3. The method according to claim 1 wherein the reference gene expression profiles are obtained from primary tumor tissue from prostate cancer patients.

4-39. (canceled)

40. A method of treating prostate cancer in a subject in need thereof, said method comprising:
(a) using the method of claim 1 for classifying a sample, said sample comprising tumor-derived material from the subject diagnosed with prostate cancer, as one of the prostate cancer subtypes MetA, MetB and MetC; and
(b) administering a prostate cancer treatment to the subject; wherein
(i) if the sample is classified as a MetA subtype, the subject is administered androgen deprivation therapy and/or androgen receptor targeting therapy, preferably as the sole anti-cancer therapy against the prostate cancer;
(ii) if the sample is classified as a MetB subtype, the subject is administered (I) androgen deprivation therapy and/or androgen receptor targeting therapy, in combination with (II) chemotherapy and/or therapy using DNA repair inhibitors; and
(iii) if the sample is classified as a MetC subtype, the subject is administered (I) androgen deprivation therapy and/or androgen receptor targeting therapy, in combination with (II) therapy targeting the tumor micro-environment.

41. The method according to claim 40 wherein the said tumor-derived material comprises tumor cells.

42. A kit for classifying a prostate cancer subtype, said kit comprising
(a) reagents for detecting at least 10 biomarkers; and
(b) instructions for using the said reagents in an assay for detecting the presence of the at least 10 biomarkers;
wherein the biomarkers are selected from one of the following groups:
(i) biomarkers for the detection of subtype MetA, selected from the group consisting of ACAA1, ACP6, ACPP, ACSS1, ALDH1A3, ALDH6A1, ATP2C1, C9orf91, CANT1, CDH1, CDS1, COG3, CPNE4, CRELD1, CTBS, DHRS7, ENTPD5, ENTPD6, FAM174B, FICD, GABARAPL2, GREB1, GTF3C1, H2AFJ, HPN, IVD, KIAA0251, KLK2, KLK3, LOC124220, LOC642299, LOC731999, NAAA, NECAB3, NWD1, PLA2G4F, PPAP2A, PSD4, REXO2, RNF41, SCFDL, SCCPDH, SEC22C, SEC23B, SECISBP2L, SELT, SLC25A17, SLC35A3, SLC37A1, SLC39A6, SLC4A4, SLC9A2, SLC9A3R1, STEAP2, SUOX, TSPAN1, WASF3, VIPR1, VPS54, and XBP1;
(ii) biomarkers for the detection of subtype MetB, selected from the group consisting of ASPM, BUB1, C12orf48, C16orf75, C17orf53, C1orf135, C6orf173, CCNA2, CCNB1, CCNB2, CDC2, CDC20, CDC451, CDCA3, CDCA4, CENPF, CENPL, CKSIB, CKS2, DDX39, DEK, ECT2, FAM83D, GAS2L3, HMGB2, KIF11, KIF15, KIF20A, KIF23, KIFC1, LIN9, LOC399942, LOC643287, LSM2, MAD2L1, MCM10, MCM2, MCM7, MDC1, MEST, MSH6, NCAPG, NUSAP1, OIP5, PHF16, PSRC1, PTMA, PTTG3P, RACGAP1, RFC5, STIL, STMN1, TOP2A, TPX2, TTK, TUBB, UBE2C, UNG, USP1, and ZNF250; and
(iii) biomarkers for the detection of subtype MetC, selected from the group consisting of AEBP1, AP1S2, ARHGAP23, ARHGEF6, BMP1, C10orf54, C1orf54, C1QTNF5, CAV1, CD93, CDH5, CLDN5, CLIP3, COL6A2, COL6A3, COX7A1, CYYR1, DDR2, DPYSL2, ENG, FAM176B, FERMT2, FGD5, FNDC1, FXYD5, GAS6, GIMAP4, GIMAP8, GJA4, GYPC, ICAM2, IGFBP4, ITGA5, JAM3, KIAA1602, LOC730994, LYL1, MGC4677, MSN, NAALADL1, NINJ2, PARVG, PDGFRB, PECAM1, PLCG2, PLCL2, RAB31, RASIP1, SH2B3, SH3KBP1, SLIT3, SRPX2, STAB1, STOM, TCF4, TEK, TPM2, TPST2, UBTD1, and VAMP5.
